SEQUENCE LISTING (21 sequences)

SEQ ID NO: 1 - DNA, 1878 bp, Artificial Sequence
  Synthetic sequence, codon optimized gene cry1Da sequence
atggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgtgagggccttctccgactgggagaaggacccgaccaacccggccctgagggaggagatgaggatccagttcaacgacatgaactccgccctgatcaccgccatcccgctgttcagggtgcagaactacgaggtggccctgctgtccgtgtacgtgcaggccgccaacctgcacctgtccatcctgagggacgtgtccgtgttcggcgagaggtggggctacgacaccgccaccatcaacaacaggtactccgacctgacctccctgatccacgtgtacaccaaccactgcgtggacacctacaaccagggcctgaggaggctggagggcaggttcctgtccgactggatcgtgtacaacaggttcaggaggcagctgaccatctccgtgctggacatcgtggccttcttcccgaactacgacatcaggacctacccgatccagaccgccacccagctgaccagggaggtgtacctggacctgccgttcatcaacgagaacctgtccccggccgcctcctacccgaccttctccgccgccgagtccgccatcatcaggtccccgcacctggtggacttcctgaactccttcaccatctacaccgactccctggccaggtacgcctactggggcggccacctggtgaactccttcaggaccggcaccaccaccaacctgatcaggtccccgctgtacggcagggagggcaacaccgagaggccggtgaccatcaccgcctccccgtccgtgccgatcttcaggaccctgtcctacatcaccggcctggacaactccaacccggtggccggcatcgagggcgtggagttccagaacaccatctccaggtccatctacaggaagtccggcccgatcgactccttctccgagctgccgccgcaggacgcctccgtgtccccggccatcggctactcccacaggctgtgccacgccaccttcctggagaggatctccggcccgaggatcgccggcaccgtgttctcctggacccacaggtccgcctccccgaccaacgaggtgtccccgtccaggatcacccagatcccgtgggtgaaggcccacaccctggcctccggcgcctccgtgatcaagggcccgggcttcaccggcggcgacatcctgaccaggaactccatgggcgagctgggcaccctgagggtgaccttcaccggcaggctgccgcagtcctactacatcaggttcaggtacgcctccgtggccaacaggtccggcaccttcaggtactcccagccgccgtcctacggcatctccttcccgaagaccatggacgccggcgagccgctgacctccaggtccttcgcccacaccaccctgttcaccccgatcaccttctccagggcccaggaggagttcgacctgtacatccagtccggcgtgtacatcgacaggatcgagttcatcccggtgaccgccaccttcgaggccgagtacgacctggagagggcccagaaggtggtgaacgccctgttcacctccaccaaccagctgggcctgaagaccgacgtgacctaa

SEQ ID NO: 2 - DNA, 1878 bp, Bacillus thuringiensis
atggaaataaataatcaaaaccaatgtgtgccttacaattgtttaagtaatcctaaggagataatattaggcgaggaaaggctagaaacagggaatactgtagcagacatttcattagggcttattaattttctatattctaattttgtaccaggaggaggatttatagtaggtttactagaattaatatggggatttatagggccttcgcaatgggatatttttttagctcaaattgagcaattgattagtcaaagaatagaagaatttgctaggaatcaggcaatttcaagattggaggggctaagcaatctttataaggtctatgttagagcgtttagcgactgggagaaagatcctactaatcctgctttaagggaagaaatgcgtatacaatttaatgacatgaatagtgctctcataacggctattccactttttagagttcaaaattatgaagttgctcttttatctgtatatgttcaagccgcaaacttacatttatctattttaagggatgtttcagttttcggagaaagatggggatatgatacagcgactatcaataatcgctatagtgatctgactagccttattcatgtttatactaaccattgtgtggatacgtataatcagggattaaggcgtttggaaggtcgttttcttagcgattggattgtatataatcgtttccggagacaattgacaatttcagtattagatattgttgcgttttttccaaattatgatattagaacatatccaattcaaacagctactcagctaacgagggaagtctatctggatttaccttttattaatgaaaatctttctcctgcagcaagctatccaaccttttcagctgctgaaagtgctataattagaagtcctcatttagtagactttttaaatagctttaccatttatacagatagtctggcacgttatgcatattggggagggcacttggtaaattctttccgcacaggaaccactactaatttgataagatcccctttatatggaagggaaggaaatacagagcgccccgtaactattaccgcatcacctagcgtaccaatatttagaacactttcatatattacaggccttgacaattcaaatcctgtagctggaatcgagggagtggaattccaaaatactataagtagaagtatctatcgtaaaagcggtccaatagattcttttagtgaattaccacctcaagatgccagcgtatctcctgcaattgggtatagtcaccgtttatgccatgcaacatttttagaacggattagtggaccaagaatagcaggcaccgtattttcttggacacaccgtagtgccagccctactaatgaagtaagtccatctagaattacacaaattccatgggtaaaggcgcatactcttgcatctggtgcctccgtcattaaaggtcctggatttacaggtggagatattctgactaggaatagtatgggcgagctggggaccttacgagtaaccttcacaggaagattaccacaaagttattatatacgtttccgttatgcttcggtagcaaataggagtggtacatttagatattcacagccaccttcgtatggaatttcatttccaaaaactatggacgcaggtgaaccactaacatctcgttcgttcgctcatacaacactcttcactccaataaccttttcacgagctcaagaagaatttgatctatacatccaatcgggtgtttatatagatcgaattgaatttataccggttactgcaacatttgaggcagaatatgatttagaaagagcgcaaaaggtggtgaatgccctgtttacgtctacaaaccaactagggctaaaaacagatgtgacgtaa

SEQ ID NO: 3 - protein, 625 aa, Artificial Sequence
  Synthetic sequence, amino acid sequence translated from the codon-optimized sequence of the cry1Da gene
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAASYPTFSAAESAIIRSPHLVDFLNSFTIYTDSLARYAYWGGHLVNSFRTGTTTNLIRSPLYGREGNTERPVTITASPSVPIFRTLSYITGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVT

SEQ ID NO: 4 - DNA, 1508 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagg

SEQ ID NO: 5 - DNA, 253 bp, Agrobacterium sp.
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 6 - DNA, 654 bp, Cauliflower mosaic virus
acatcaatccacttgctttgaagacgtggttggaacgtcttctttttccacgatgctcctcgtgggtgggggtccatctttgggaccactgtcggtagaggcatcttgaacgatagcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgac

SEQ ID NO: 7 - DNA, 246 bp, Tobacco etch
ggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatc

SEQ ID NO: 8 - DNA, 553 bp, Streptomyces hygroscopicus
gagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcat

SEQ ID NO: 9 - DNA, 577 bp, Glycine max
acgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcggtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgtt

SEQ ID NO: 10 - DNA, 3663 bp, Artificial Sequence
  Synthetic sequence, ubi-cry1Da-nos artificial construct
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggggatccgagctcatggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgtgagggccttctccgactgggagaaggacccgaccaacccggccctgagggaggagatgaggatacagttcaacgacatgaactccgccctgatcaccgccatcccgctgttcagggtgcagaactacgaggtggccctgctgtccgtgtacgtgcaggccgccaacctgcacctgtccatcctgagggacgtgtccgtgttcggcgagaggtggggctacgacaccgccaccatcaacaacaggtactccgacctgacctccctgatccacgtgtacaccaaccactgcgtggacacctacaaccagggcctgaggaggctggagggcaggttcctgtccgactggatcgtgtacaacaggttcaggaggcagctgaccatctccgtgctggacatcgtggccttcttcccgaactacgacatcaggacctacccgatccagaccgccacccagctgaccagggaggtgtacctggacctgccgttcatcaacgagaacctgtccccggccgcctcctacccgaccttctccgccgccgagtccgccatcatcaggtccccgcacctggtggacttcctgaactccttcaccatctacaccgactccctggccaggtacgcctactggggcggccacctggtgaactccttcaggaccggcaccaccaccaacctgatcaggtccccgctgtacggcagggagggcaacaccgagaggccggtgaccatcaccgcctccccgtccgtgccgatcttcaggaccctgtcctacatcaccggcctggacaactccaacccggtggccggcatcgagggcgtggagttccagaacaccatctccaggtccatctacaggaagtccggcccgatcgactccttctccgagctgccgccgcaggacgcctccgtgtccccggccatcggctactcccacaggctgtgccacgccaccttcctggagaggatctccggcccgaggatcgccggcaccgtgttctcctggacccacaggtccgcctccccgaccaacgaggtgtccccgtccaggatcacccagatcccgtgggtgaaggcccacaccctggcctccggcgcctccgtgatcaagggcccgggcttcaccggcggcgacatcctgaccaggaactccatgggcgagctgggcaccctgagggtgaccttcaccggcaggctgccgcagtcctactacatcaggttcaggtacgcctccgtggccaacaggtccggcaccttcaggtactcccagccgccgtcctacggcatctccttcccgaagaccatggacgccggcgagccgctgacctccaggtccttcgcccacaccaccctgttcaccccgatcaccttctccagggcccaggaggagttcgacctgtacatccagtccggcgtgtacatcgacaggatcgagttcatcccggtgaccgccaccttcgaggccgagtacgacctggagagggcccagaaggtggtgaacgccctgttcacctccaccaaccagctgggcctgaagaccgacgtgacctaagtcgacggatccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 11 - DNA, 5711 bp, Artificial Sequence
  Synthetic sequence
acgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcggtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggtagaggcatcttgaacgatagcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggggatccgagctcatggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgtgagggccttctccgactgggagaaggacccgaccaacccggccctgagggaggagatgaggatacagttcaacgacatgaactccgccctgatcaccgccatcccgctgttcagggtgcagaactacgaggtggccctgctgtccgtgtacgtgcaggccgccaacctgcacctgtccatcctgagggacgtgtccgtgttcggcgagaggtggggctacgacaccgccaccatcaacaacaggtactccgacctgacctccctgatccacgtgtacaccaaccactgcgtggacacctacaaccagggcctgaggaggctggagggcaggttcctgtccgactggatcgtgtacaacaggttcaggaggcagctgaccatctccgtgctggacatcgtggccttcttcccgaactacgacatcaggacctacccgatccagaccgccacccagctgaccagggaggtgtacctggacctgccgttcatcaacgagaacctgtccccggccgcctcctacccgaccttctccgccgccgagtccgccatcatcaggtccccgcacctggtggacttcctgaactccttcaccatctacaccgactccctggccaggtacgcctactggggcggccacctggtgaactccttcaggaccggcaccaccaccaacctgatcaggtccccgctgtacggcagggagggcaacaccgagaggccggtgaccatcaccgcctccccgtccgtgccgatcttcaggaccctgtcctacatcaccggcctggacaactccaacccggtggccggcatcgagggcgtggagttccagaacaccatctccaggtccatctacaggaagtccggcccgatcgactccttctccgagctgccgccgcaggacgcctccgtgtccccggccatcggctactcccacaggctgtgccacgccaccttcctggagaggatctccggcccgaggatcgccggcaccgtgttctcctggacccacaggtccgcctccccgaccaacgaggtgtccccgtccaggatcacccagatcccgtgggtgaaggcccacaccctggcctccggcgcctccgtgatcaagggcccgggcttcaccggcggcgacatcctgaccaggaactccatgggcgagctgggcaccctgagggtgaccttcaccggcaggctgccgcagtcctactacatcaggttcaggtacgcctccgtggccaacaggtccggcaccttcaggtactcccagccgccgtcctacggcatctccttcccgaagaccatggacgccggcgagccgctgacctccaggtccttcgcccacaccaccctgttcaccccgatcaccttctccagggcccaggaggagttcgacctgtacatccagtccggcgtgtacatcgacaggatcgagttcatcccggtgaccgccaccttcgaggccgagtacgacctggagagggcccagaaggtggtgaacgccctgttcacctccaccaaccagctgggcctgaagaccgacgtgacctaagtcgacggatccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 12 - DNA, 20 bp, Artificial Sequence
  Synthetic oligonucleotide
accggctagagccatcccag

SEQ ID NO: 13 - DNA, 17 bp, Artificial Sequence
  Synthetic oligonucleotide
accaaccagcgaaccag

SEQ ID NO: 14 - DNA, 18 bp, Artificial Sequence
  Synthetic oligonucleotide
agaaacccacgtcatgcc

SEQ ID NO: 15 - DNA, 18 bp, Artificial Sequence
  Synthetic oligonucleotide
tgcaccatcgtcaaccac

SEQ ID NO: 16 - DNA, 22 bp, Artificial Sequence
  Synthetic oligonucleotide
ttgatgtgggttttactgatgc

SEQ ID NO: 17 - DNA, 20 bp, Artificial Sequence
  Synthetic oligonucleotide
caccttgtacaggttggaca

SEQ ID NO: 18 - DNA, 18 bp, Artificial Sequence
  Synthetic oligonucleotide
ctgcagcaagctatccaa

SEQ ID NO: 19 - DNA, 18 bp, Artificial Sequence
  Synthetic oligonucleotide
atttgaattgtcaaggcc

SEQ ID NO: 20 - DNA, 24 bp, Artificial Sequence
  Synthetic oligonucleotide
aaaatggagataaataatcaaaac

SEQ ID NO: 21 - DNA, 27 bp, Artificial Sequence
  Synthetic oligonucleotide
aaactattcttccataaggagtaattc

(19) United States
(12) Patent Application Publication
(10) Pub. No.: US 2021/0340558 A1
(43) Pub. Date: Nov. 4, 2021

(54) CODON-OPTIMISED CRYIDA NUCLEIC ACID MOLECULE, NUCLEIC ACID CONSTRUCT, VECTOR, HOST CELL, PLANT CELL, TRANSGENIC PLANT, METHOD FOR TRANSFORMING A CELL, METHOD FOR PRODUCING A TRANSGENIC PLANT, METHOD FOR CONTROLLING INVERTEBRATE PESTS OF CROP PLANTS, AND USES OF THE NUCLEIC ACID MOLECULE

(71) Applicants: Empresa Brasileira de Pesquisa Agropecuária - Embrapa, Brasília - DF (BR); Helix Sementes e Mudas Ltda., Rio Claro -SP (BR)

(72) Inventors: Newton PORTILHO CARNEIRO, Sete Lagoas (BR); Fernando HERCOS VALICENTE, Sete Lagoas (BR); Andréa ALMEIDA CARNEIRO, Sete Lagoas (BR); Roberto WILLIANS NODA, Sete Lagoas (BR); Meire DE CÁSSIA ALVES, Sete Lagoas (BR); Beatriz DE ALMEIDA BARROS, Sete Lagoas (BR)

(73) Assignees: Empresa Brasileira de Pesquisa Agropecuária - Embrapa, Brasília - DF (BR); Helix Sementes e Mudas Ltda., Rio Claro -SP (BR)

(21) Appl. No.: 17/053,638

(22) PCT Filed: Apr. 30, 2019
(86) PCT No.: PCT/BR2019/050158

§ 371 Date: Jun. 23, 2021

(30) Foreign Application Priority Data
May 7, 2018 (BR) 10 2018 009263 4

(51) Int. Cl. C12N 15/82; C07K 14/325

(52) U.S. Cl. CPC C12N 15/8286; C07K 14/325

(57) ABSTRACT

The present invention relates to new codon-optimized cry1Da nucleic acid molecules from a gene sequence isolated from bacterium Bacillus thuringiensis. These molecules are used in the preparation of nucleic acid constructs, vectors and host cells, allowing the production of transgenic plants, such as corn, resistant to invertebrate pests, such as insects from the order Lepidoptera, particularly Spodoptera frugiperda (Noctuidae, Lepidoptera) and Diatrea saccharalis (Crambidae, Lepidoptera). Plant cells and transgenic plants comprising the molecules or constructs of the invention are also objects of the present invention. In particular, the transgenic plants according to the present invention are able to control caterpillars of the cited species that have become resistant to plants containing the cry1F gene. In addition, the present invention relates to a method for transforming a cell, a method of controlling invertebrate pests in crop plants and uses of nucleic acid molecules or constructs in the production of transgenic plants and for controlling invertebrate pests.

### FIELD OF THE INVENTION

The present invention relates to new codon-optimized cry1 Da nucleic acid molecules from a gene sequence isolated from bacterium Bacillus thuringiensis. These molecules are used in the preparation of nucleic acid constructs, vectors and host cells, allowing the production of transgenic plants, such as corn, resistant to invertebrate pests, such as insects from the order Lepidoptera, particularly Spodoptera frugiperda (Noctuidae, Lepidoptera) and Diatrea saccharalis (Crambidae, Lepidoptera). Transgenic plant cells and plants comprising the molecules or constructs of the invention are also objects of the present invention. In particular, the transgenic plants according to the present invention are able to control caterpillars of the cited species that have become resistant to plants containing the cry1F gene. In addition, the present invention relates to a method for transforming a cell, a method of controlling invertebrate pests in crop plants and uses of nucleic acid molecules or constructs in the production of transgenic plants and for controlling invertebrate pests.

### BACKGROUND OF THE INVENTION

Consistent advances in genetic engineering techniques have enabled the development of transgenic plants of commercial importance, containing the heterologous genes of interest, which can confer desirable traits to such plants. From among the genes of interest are genes that confer on plants resistance to herbicides, environmental stresses, and invertebrate pests, for example.

In the context of genes that code for proteins useful for controlling invertebrate pests, cry gene, derived from the Gram-positive bacterium Bacillus thuringiensis (Bt), can be mentioned. Said bacteria, which occurring naturally in several habitats, including the soil, phylloplane, grain residues, dust, water, plant matter and insects, has the innate characteristic of forming protein crystals during the stationary and/or sporulation phase. Protein crystals or delta-endotoxins, representing 20 to 30% of the total cell protein (Boucias & Pendland, 1998), have specific insecticidal properties and can be of various shapes, such as: bipyramidal, spherical, rectangular, cuboid and irregular. Bipyramidal crystals have a higher frequency of toxicity than crystals of other shapes, acting particularly against lepidopterans.

The mechanism of action of Cry proteins involves, in general, the solubilization of crystals in the midgut of insect larvae, the action of proteases on pro-toxins, adherence of active toxins to midgut receptors and the insertion of said active toxins into the apical cell membrane, creating ion channels or pores (cytolysis).

An advantage from the use of Cry proteins is the activity thereof against various insect species, being considered safe in relation to other organisms, such as mammals. Another advantage is the relative to the specificity thereof towards pest insects from different crops. Several cry genes are known. Cry1, cry2 and cry9 genes are generally active against lepidopterans; cry2, cry4A, cry10, cry11, cry17, cry19, cry24, cry25, cry27, cry29, cry30, cry32, cry39 and cry40 genes are generally active against dipterans; cry3, cry7 and cry8 genes are generally active against coleopterans; and cry5, cry12, cry13 and cry14 genes are generally active against nematodes.

Bt-based formulations available on the market represent a high percentage of sales of biopesticides and have been used for over 40 years for the control of pests from the orders Lepidoptera and Diptera. Production of the first transgenic plants containing cry genes did not present satisfactory results. In general, the expression levels of native genes were lower than those necessary to provide adequate protection against the target species in the field. Such a low concentration of Cry proteins was due to, among other factors, an incompatibility between codons of the gene donor species (Bt bacteria) and the gene recipient species (plants of interest).

It is known that different species use preferred codons, in particular for coding proteins, and these codon variations can negatively affect gene expression in the context of transgenics (Gustafsson, 2004). For example, in maize, codon AAG is used preferentially over codon AAA for amino acid lysine (Liu, 2009). Due to this unique characteristic of different groups of organisms, insertion of native B. thuringiensis cry genes into plants leads to low expression of the Cry protein of interest.

Moreover, bacterial genes have a low C+G content, in contrast to plant genes (Cambel & Gowri, 1990; Murray et al., 1989). A+T-rich bacterial nucleotide sequences can be recognized by plants as splice sites (Liu, 2009), polyadenylation signals (Joshi, 1987; Diehn et al., 1998) or elements of RNA destabilization, such as ATTTA (Ohme-Takagi et al., 1993). Therefore, to increase the expression of a Bt bacterium cry gene in the recipient organism, the gene must be “recoded”, not only to adapt it to the preferred amino acid codons, but also to bring them closer to the G+C content of the recipient organism.

Genetic manipulation of cry genes can be promising to improve the efficiency and cost/benefit relationship of bioinsecticides and transgenic plants expressing these genes. Different Bt isolates can show a very wide range of toxic activity against the same target species, and one isolate can be very active against one species and virtually inactive against another (Jarret & Burges, 1982). Some combinations of Cry proteins have even shown synergistic toxicity towards lepidopterans. These authors reported that bioassays have shown synergism between Cry1Aa and Cry1Ac proteins, while the mixture of Cry1Aa and Cry1Ab exhibited antagonism towards the control of Lymantria dispar.

Considering the diversity of responses achieved by combining Cry insecticidal proteins and pest insects, as well as the importance of controlling such insects in crop plants, studies intended to better elucidate the decisive characteristics for obtaining satisfactory insecticidal effects as well as those aimed at the development of new transgenic plants resistant to insects are of major importance.

In previous studies, Bt isolates were tested against Spodoptera frugiperda or fall armyworm in vitro and molecular characterization of the most efficient ones was performed. Isolation of Bt strains was confirmed by means of a phase contrast microscope by observing the protein crystals. Bioassays to assess the toxicity of Bt strains were then carried out by exposing two-day-old larvae reared on an artificial diet to a suspension of spores and crystals. The caterpillars (25 larvae/bioassay/strain) were placed in disposable plastic containers (50 mL) at a temperature of 27° C., relative moisture of 70%, and a 14 h/10 h photophase. Strains were considered efficient when mortality was higher than 75%.

The literature mentions that 13 B. thuringiensis serovarieties were tested in S. frugiperda larvae and reports that serovars (sv) galleriae, aizawai and tolworthi caused mortality higher than 90%. This data was partially confirmed by the results achieved at Embrapa Milho e Sorgo laboratory, where sv tolworthi killed over 95%. However, sv galleriae and aizawai did not cause mortality greater than 15%. Bohorova et al. (1996) tested more than 400 strains against the main maize crop pests and results have shown that 99% of isolates caused mortality below 50%. These numbers are important as they show the difficulty in finding Bt isolates efficient for controlling fall armyworm. This difficulty in controlling S. frugiperda caterpillars with Cry proteins is confirmed by Baum (1999), who claims that there may be variation within the same genus.

Currently, the Polymerase Chain Reaction (PCR) is one of the most widely used molecular techniques to characterize B. thuringiensis strains (Carozzi et al. 1991, Cerón et al. 1994, Cerón et al. 1995, Bravo et al. 1998, Valicente et al. 2000). From among the most efficient strains from Embrapa Milho e Sorgo collection, most isolates carried cry1Ab and cry1E genes, some of them carried cry1B, cry1D, cry1Fb genes and only a single strain so far carries cry1C (Valicente et al. 2000, Valicente 2003). Bravo et. al 1998 made a characterization of cry genes from a Mexican collection of B. thuringiensis and found that cry1D and cry1C genes were the most toxic for S. frugiperda and S. exigua caterpillars.

For the production of transgenic plants, such as, for example, Bt transgenic maize, three basic requirements are necessary: (i) in vitro regeneration of the plant tissue to be transformed; (ii) the methodology for inserting the cry gene into the plant genome and (iii) the gene construct with cry genes and selection markers.

The development of cell and tissue culture techniques combined with recombinant DNA technology has considerably expanded the potential for using in vitro culture methods for the production of transgenic maize plants. As part of this process, the establishment of plant regeneration systems from somatic cells is a prerequisite of major importance. The most used methodology for maize regeneration in vitro is somatic embryogenesis, which has the advantage of producing a bipolar structure that can, theoretically, be germinated and regenerated in a single step.

In particular, in maize, plant regeneration via somatic embryogenesis can be made from Type I or Type II calluses (Armstrong & Green, 1985). Type I calluses are compact, yellow or white and usually capable of regenerating plants while Type II calluses are soft, friable and highly embryogenic. Type II callus-forming cultures grow rapidly, can be maintained for a long period of time and form a large number of easily regenerable somatic embryos (Vasil, 1987).

Although Type II calluses are the most efficient in the production of transgenic maize plants, Type I calluses can also be used. Occurrence of friable Type II embryogenic calluses is not so common, only a limited number of maize genotypes is able to express such a phenotype in a culture medium, particularly the A188 strain (Armstrong & Green, 1985) and the Hill hybrid (Armstrong et al. 1991).

With the improvement of in vitro culture methodologies and, particularly changes in the culture media composition and ratios and doses of growth regulators, the regeneration of an increasing number of genotypes has become possible (Novac et al., 1983; Rapela, 1985; Duncan et al., 1985; Phillips et al., 1988; Prioli & Silva, 1989; Lupotto, 2004; Petrillo et al., 2008). However, most of these genotypes forms only Type I compact calluses.

Although most of maize genotypes capable of regenerating plants is adaptable to Temperate climates, Tropical climate-adapted genotypes capable of regeneration have also been identified (Carvalho et al., 1994; Bohorova et al., 1995, Santos-Serejo & Aguiar-Perecin, 2000; Petrillo et al., 2008), which indicates the possibility of manipulating elite Tropical genotypes via genetic transformation. Immature zygotic embryos are the preferred explants for the generation of embryogenic crops and the production of transgenic maize plants.

The different methods of genetic plant transformation can be divided into two main groups: indirect and direct methods. Indirect genetic transformation uses a bacterium, Agrobacterium tumefaciens, to introduce the gene of interest into the plant genome.

For several years, Agrobacterium transformation of monocots had a very low efficiency. However, recently, this gene transfer methodology has become the method of choice for this group of plants. This method uses a natural gene transfer system developed by Agrobacterium. Agrobacterium is a soil bacterium capable of causing plant tumors in the infection site. These tumors result from the presence of Ti plasmid or tumor-inducing plasmid in the bacterial cell. Ti plasmid is a large circular molecule (200 to 800 kb) of double-stranded DNA that can replicate independently of the Agrobacterium tumefaciens genome (Gelvin, 2003). Located on the Ti plasmid are two important regions for the transfer of genes from the bacteria to the plant, the T-DNA region and the vir region. T-DNA regions of wild-type plasmids contain genes that control the production of opines and hormones, such as auxin and cytokinin, by the plant cell. Opines are amino acids used only by Agrobacterium as a source of carbon and nitrogen, while hormones are responsible for inducing plant tumors. T-DNA is between 10 and 30 kb and its ends are delimited by two highly homologous 25-pb sequences, designated as right and left ends. Wild-type Agrobacterium transfers its T-DNA through plant cell membranes and incorporates it into the plant genomic DNA. Processing of T-DNA and its transfer into the plant cell is due in large part to the virulence of proteins encoded in the vir region (Gelvin, 2003).

To enable the use of Agrobacterium in biotechnological processes of gene transfer into plants, tumor-causing T-DNA endogenous genes have to be inactivated and the exogenous genes, the gene of interest (GDI) and the selection marker gene (GMS) have to be inserted between the right and left ends of T-DNA. The resulting recombinant plasmid is placed into Agrobacterium once again to be transferred to plant cells (Gelvin, 2003). Transformed tissues or cells can be used for the regeneration of transgenic plants (Schafer et al., 1987; Hiei et al., 1994; Ishida et al., 1996).

Ti plasmid is difficult to manipulate for being too large. Therefore, binary vectors were created (Bevan, 1984), which are smaller and capable of multiplying both in Agrobacterium and E. coli and are easy to manipulate in the laboratory. These vectors have an artificial T-DNA, in which different transgenes can be inserted as well as an origin of replication compatible with Ti in Agrobacterium. Binary vectors are introduced into disarmed Agrobacterium, that is, in Agrobacterium carrying Ti plasmids that have had the T-DNA region removed. Disarmed Agrobacterium Ti still has the virulence region (vir), and its genes are capable of acting in trans to transfer recombinant T-DNA from the binary vector (Gelvin, 2003).

Agrobacterium tumefaciens is an excellent system for introducing genes into plant cells since: (i) DNA can be introduced into all plant tissues, which eliminates the need for protoplast production; and, (ii) T-DNA integration is a relatively accurate process. The DNA region to be transferred is defined by the flanking sequences, right and left ends. Occasionally, rearrangements occur, but in most cases the region is inserted intact into the plant genome. Integrated T-DNAs usually show consistent genetic maps and adequate segregation. Furthermore, the characters introduced by this route were shown to be stable over many generations of crosses. Such a stability is critical when the generated transgenic plants are to be commercialized (Hiei et al., 1994; Ishida et al., 1996).

In particular, for maize, the Agrobacterium technique has been reported to result in high efficiency with a high number of events containing a single one or a low number of copies of the transgene in the genome as compared to biobalistics (Ishida et al., 1996; Zhao et al. 2001; Gordon-Kamm et al. 1990; Frame et al. 2002; Lupotto et al. 2004; Huang and Wei 2005; Ishida et al. 2007).

Transgenes, that is, genes that are inserted via molecular biology techniques, are basically constituted by the coding region of the gene of interest or the selection marker gene and regulatory sequences of gene expression. The gene of interest (GDI) and the selection marker gene (GMS) are coding sequences or ORF (Open Reading Frame) of a certain protein that, when expressed, define a trait of interest. GMS serves to identify and select cells having the heterologous DNA integrated into the genome. They are fundamental for the development of plant transformation technologies, since the process of transferring a transgene into a recipient cell and its integration into the genome is very inefficient in most experiments, so that the chances of recovering transgenic lines without selection are generally very low. Currently, the most commonly used GMS for the production of transgenic plants, such as maize, are those that confer tolerance to herbicides. From among them, bar genes isolated from Streptomyces hygroscopicus and pat gene isolated from Streptomyces viridochromogenes, both encoding the phosphinothricin acetyltransferase enzyme (Pat) (De Block et al., 1989) are frequently used (Gordon-Kamm et al. 1990; Zhao et al. 2001, Ishida et al 2007).

Both the nucleotide sequence that codes for the protein of interest, and the one that codes for the protein used in the selection of transgenic calluses is accompanied by regulatory sequences, such as promoters and terminators, which are responsible for controlling gene expression.

Promoters are DNA sequences usually present at the 5′ ends of a coding region, used by RNA polymerase and transcription factors to initiate the gene transcription process (Buchanan et al., 2000). The 35S viral promoter isolated from cauliflower mosaic virus (CaMV35S) is one of the most commonly used promoters to target a high level of constitutive expression in plants (Odell et al., 1985), however it does not function in monocots as efficiently as it does in dicots. Currently, the most widely used promoter to target the expression of a constitutive protein in maize is the promoter isolated from maize ubiquitin gene Ubi1 (Christensen & Quail, 1996).

The 3′ UTR regions, also known as terminator regions, are used to signal the end of transcription (Lessard et al. 2002), preventing the production of chimeric RNA molecules and accordingly, the formation of new proteins, if the polymerase complex continues to transcribe beyond its end signal. The 3′ UTR sequences most used in gene constructs for maize transformation include 3′ regions from Agrobacterium nopaline synthase gene (nos) (Depicker et al., 1982), CaMV35S (Frame et al., 2002), and those from potato proteinase inhibitor gene pinII (An et al., 1989).

Although for some of the aforementioned techniques emphasis has been given to the results achieved in maize, the techniques for obtaining transgenic plants are generally known to the person skilled in the art and may vary depending on the plant of interest to be used. The skilled person can, without undue experimentation and based on his general knowledge and the available scientific literature, easily adapt the preferred methodologies reported in the instant document in order to obtain several transgenic plants.

Despite the increased scientific knowledge on the role of Cry proteins in the insecticidal activity against crop pests, the approaches so far have not been found promising enough since for some crops, there is marked resistance of the pests to those proteins, so new strategies are constantly needed.

Particularly for maize crops, it has been found that transgenic plants existing in the Brazilian market, such as those bearing cry1F gene in their genome (for example, Herculex HX), no longer adequately control some of the pest species, such as populations of Spodoptera frugiperda and Diatrea saccharalis caterpillars.

In this sense, the present invention was developed to satisfy the needs of the prior art, which discloses a new codon-optimized cry1 Da nucleic acid molecule, as well as the generation of transgenic maize plants expressing this gene efficiently, hence obtaining effective control of several species of invertebrate pests, including those that were resistant to other transgenic maize crops at the time of the present invention (for example, Herculex HX). Currently, the Brazilian market has no transgenic maize plant expressing Cry1 Da protein isolated from Bacillus thuringiensis for the control, for example, of Spodoptera frugiperda and considering the transgenic maize plants existing in the state of the art, that those plants or their insecticidal gene inserts would not be expected to lead, with a reasonable expectation of success, to the efficient control of different pest species or even of different populations of that species.

cry1 Da sequences, as disclosed by the present invention, and having optimized G+C content, led to highly efficient pest control, having even been effective against resistant pests of maize crops existing at the time of the present invention, solving one problem of the state of the art. As previously discussed, even if an eventual protein is equal or similar to the already reported Cry1 Da proteins, the nucleotide sequences, as disclosed herein, ensured improved expression and made it possible for one to achieve exceptional results, as exemplified herein, which could not be expected, with a reasonable expectation of success in view of the teachings of the state of the art.

In this sense, the present invention represents a major improvement over the state of the art.

### BRIEF DESCRIPTION OF THE SEQUENCES

SEQ ID NO: 1 refers to the codon-optimized nucleic acid sequence of cry1 Da gene of the present invention.

SEQ ID NO: 2 refers to the nucleic acid sequence of cry1 Da gene isolated from Bacillus thuringiensis, from which the optimization was carried out.

SEQ ID NO: 3 refers to the amino acid sequence translated from SEQ ID NO: 1 or SEQ ID NO: 2 (Cry1 Da protein).

SEQ ID NO: 4 refers to the nucleic acid sequence of maize ubiquitin gene (ubi) promoter region used in the construct of the present invention.

SEQ ID NO: 5 refers to the nucleic acid sequence of the 3′ UTR terminator region of Agrobacterium nopaline synthase gene (nos) used in the construct of the present invention.

SEQ ID NO: 6 refers to the nucleic acid sequence of the duplicated CaMV 35S gene promoter region of cauliflower mosaic virus used in the construct of the present invention.

SEQ ID NO: 7 refers to the nucleic acid sequence of the enhancer translational region of Tobacco etch (tev) virus used in the construct of the present invention.

SEQ ID NO: 8 refers to the nucleic acid sequence of the region encoding the phosphinothricin acetyltransferase selection gene (bar) from Streptomyces hygroscopicus used in the construct of the present invention.

SEQ ID NO: 9 refers to the nucleic acid sequence of the 3′ UTR terminator region of Tvsp gene that codes for soybean vegetative storage protein used in the construct of the present invention.

SEQ ID NO: 10 refers to the nucleic acid sequence of the nucleic acid construct of the present invention (comprising maize ubiquitin gene promoter (ubi) (SEQ ID NO: 4), the codon-optimized coding sequence of the present invention (SEQ ID NO: 1) and 3′UTR terminator sequence of nopaline synthase gene (nos)) (SEQ ID NO: 5)—UBI::cry1 Da::NOS.

SEQ ID NO: 11 refers to the nucleic acid sequence of the nucleic acid construct of the present invention, in its entirety, comprising the (3′ UTR terminator region of Tvsp gene (SEQ ID NO: 9); a region coding for phosphinothricin acetyltransferase (bar) selection gene (SEQ ID NO: 8), a translational enhancer region (tev) (SEQ ID NO: 7); a promoter region of duplicated CaMV 35S gene (SEQ ID NO: 6), an ubiquitin gene promoter region (ubi) (SEQ ID NO: 4); a codon-optimized nucleic acid sequence of cry1 Da gene of the present invention (SEQ ID NO: 1); and a 3′ UTR terminator region of nopaline synthase gene (nos)) (SEQ ID NO: 5)).

SEQ ID NO: 12 refers to the 5′-3′ nucleic acid sequence of primer U4 used in gene construct cloning experiments.

SEQ ID NO: 13 refers to the 5′-3′ nucleic acid sequence of primer U1 used in the gene construct cloning experiments.

SEQ ID NO: 14 refers to the 5′-3′ nucleic acid sequence of forward primer BAR used in gene construct cloning experiments.

SEQ ID NO: 15 refers to the 5′-3′ nucleic acid sequence of reverse primer BAR used in gene construct cloning experiments.

SEQ ID NO: 16 refers to the 5′-3′ nucleic acid sequence of forward primer Ubi used in gene construct cloning experiments.

SEQ ID NO: 17 refers to the 5′-3′ nucleic acid sequence of reverse primer Cry1 Da used in gene construct cloning experiments.

SEQ ID NO: 18 refers to the 5′-3′ nucleic acid sequence of forward cry1 Da gene primer used in gene selection experiments.

SEQ ID NO: 19 refers to the 5′-3′ nucleic acid sequence of reverse cry1 Da gene primer used in gene selection experiments.

SEQ ID NO: 20 refers to the 5′-3′ nucleic acid sequence of forward cry1 Da gene primer used in experiments for isolating the full-length gene from Bt 1132C strain.

SEQ ID NO: 21 refers to the 5′-3′ nucleic acid sequence of reverse cry1 Da gene primer used in experiments for isolating the full-length gene from Bt 1132C strain.

### BRIEF DESCRIPTION OF THE FIGURES

FIG. 1 is an alignment of the codon-optimized nucleic acid sequence of cry1 Da gene of the present invention (SEQ ID NO: 1) relative to the nucleic acid sequence of cry1 Da gene isolated from Bacillus thuringiensis, from which optimization was carried out (SEQ ID NO: 2).

FIG. 2 is a drawing depicting the expression vector in pTF101.1 plants. RB/R25: T-DNA right border; LB: T-DNA left border; 2XP35S: Duplicate CaMV35S promoter of mosaic virus (Odell et al., 1985); TEV enhancer: translational enhancer of Tobacco etch virus (Gallie et al, 1995; Wilson, 1999); ORF bar: coding region of phosphinothricin acetyltransferase gene of Streptomyces hygroscopicus that confers resistance to the herbicide phosphinothricin and derivatives thereof (Thompson et al, 1987; White et al, 1990; Becker et al, 1992); Tvsp: 3′ terminator of the gene encoding the soybean vegetative storage protein (Mason et al., 1993); aadA: Shigella flexneris 2a aminoglycoside 3′-adenylyltransferase gene that confers resistance to the antibiotic spectinomycin and streptomycin (Chinault et al., 1986); pVS1: the broad range of host plasmids from Pseudomonas (Hajdukiewicz et al, 1994); multiple cloning sites formed by restriction enzymes PvuII, EcoRI, Sac, KpnI, SmaI, XmaI, BamHI, XbaI, SaII, PstI, HindIII, PstI.

FIG. 3 refers to the drawing depicting gene constructs Ubi::cry1 Da::NOS and 2×35S::bar::Tvsp inserted into Hind III and EcoRI enzyme sites of binary vector pTF101.1, between the T-DNA right and left borders.

FIG. 4 refers to a representative result of the bioassay in feeding laboratory of Spodoptera frugiperda on (A) non-transgenic maize and (B) transgenic maize comprising the codon-optimized cry1 Da nucleic acid molecule of the present invention (SEQ ID NO: 1).

FIG. 5 refers to a result representing a laboratory bioassay of feeding Diatrea saccharalis on (A) non-transgenic maize and (B) transgenic maize comprising the codon-optimized cry1 Da nucleic acid molecule of the present invention (SEQ ID NO: 1). In (C) caterpillars grown on non-transgenic maize leaves (left caterpillar) and transgenic maize of the present invention (right caterpillar) are seen.

FIG. 6 refers to a graph showing the injury score (±IC, P=0.05) caused by Spodoptera frugiperda infestation on oak scale, 1970. Treatment 1—transgenic maize comprising the codon-optimized cry1 Da nucleic acid sequence the present invention (SEQ ID NO: 1)+Cry1F resistant caterpillar population; Treatment 2=Non-transgenic L3 maize strain+Cry1F resistant caterpillar population; Treatment 3=transgenic maize comprising the codon-optimized cry1 Da nucleic acid molecule of the present invention (SEQ ID NO: 1)+Population of susceptible caterpillars; Treatment 4=Non-transgenic L3 maize strain+Population of susceptible caterpillars.

FIG. 7 is a photograph showing the result of the aforementioned injury of (A) transgenic maize comprising the codon-optimized cry1 Da nucleic acid molecule of the present invention (SEQ ID NO: 1) and (B) non-transgenic control plant infested with cry1F resistant S. frugiperda caterpillars.

### BRIEF DESCRIPTION OF THE INVENTION

The present invention relates to new codon-optimized cry1 Da nucleic acid molecules from a gene sequence isolated from bacterium Bacillus thuringiensis. These molecules are used in the preparation of nucleic acid constructs, vectors and host cells, allowing the production of transgenic plants, such as corn, resistant to invertebrate pests, such as insects from the order Lepidoptera, particularly Spodoptera frugiperda (Noctuidae, Lepidoptera) and Diatrea saccharalis (Crambidae, Lepidoptera). Plant cells and transgenic plants comprising the molecules or constructs of the invention are also objects of the present invention. In particular, the transgenic plants according to the present invention are able to control caterpillars of the cited species that have become resistant to plants containing the cry1F gene. In addition, the present invention relates to a method for transforming a cell, a method of controlling invertebrate pests in crop plants and uses of nucleic acid molecules or constructs in the production of transgenic plants and for controlling invertebrate pests.

Thus, a first object of the present invention is a codon-optimized cry1 Da nucleic acid molecule, comprising a nucleic acid sequence having at least 70% similarity with the sequence of SEQ ID NO: 1.

In a preferred embodiment of the present invention, said nucleic acid molecule comprises a nucleic acid sequence having at least 90% similarity with the sequence of SEQ ID NO: 1.

In another preferred embodiment of the present invention, said nucleic acid molecule is as defined in SEQ ID NO: 1.

Thus, a second object of the present invention is a nucleic acid construct comprising the codon-optimized cry1 Da nucleic acid molecule having at least 70% similarity with the sequence of SEQ ID NO: 1, preferably with at least 90% similarity with the sequence of SEQ ID NO: 1, more preferably being SEQ ID NO: 1.

In a preferred embodiment of the present invention, said construct further comprises a promoter sequence operably linked to said nucleic acid molecule, wherein said promoter sequence is preferably maize ubiquitin (ubi) promoter sequence.

In another preferred embodiment of the present invention, said construct further comprises a 3′ UTR terminator sequence, wherein said 3′ UTR terminator sequence is preferably nopaline synthase (nos) gene terminator sequence.

In another preferred embodiment of the present invention, said construct further comprises a selection gene operably linked to at least one promoter sequence and at least one terminator sequence, wherein the promoter sequence is preferably the duplicated CaMV 35S gene promoter sequence of cauliflower mosaic virus and the terminator sequence is preferably the Tvsp gene terminator sequence that codes for the soybean vegetative storage protein.

In another preferred embodiment of the present invention, said construct further comprises other regulatory sequences.

In another preferred embodiment of the present invention, the construct comprises the nucleic acid molecule defined as SEQ ID NO: 10.

Thus, a third object of the present invention is a vector comprising the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct of the present invention, as defined herein.

A forth object of the present invention is a host cell comprising the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct or the vector of the present invention, as defined herein.

A fifth object of the present invention is a plant cell comprising the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct or the vector of the present invention, as defined herein.

A sixth object of the present invention is a transgenic plant comprising the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct of the present invention, as defined herein.

Thus, a seventh object of the present invention is a method of transforming a cell comprising introducing into said cell the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct or the vector of the present invention, as defined herein. In a preferred embodiment of the present invention, said method comprises integrating the nucleic acid molecule into the cell genome.

An eighth object of the present invention is a method of producing a transgenic plant comprising transforming the plant cell with the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct or the vector of the present invention, as defined herein. In a preferred embodiment of the present invention, said method further comprises selecting a plant cell transformed with the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct or the vector of the present invention, as defined herein.

In another preferred embodiment of the present invention, said method further comprises the regeneration of a transgenic plant from said plant cell.

In another preferred embodiment of the present invention, said transgenic plant is resistant to crop pests, wherein the transgenic plant is preferably a monocot, preferably a corn, rice, sugarcane, sorghum, wheat or brachiaria plant.

In another preferred embodiment of the present invention, said crop pest is preferably an insect, more preferably from the order Lepidoptera, even most preferably Spodoptera frugiperda and/or Diatrea saccharalis.

A nineth object of the present invention is a method of controlling invertebrate crop pests, wherein the crop plants comprise the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1, or the nucleic acid construct of the present invention, as defined in the present document, wherein the method comprises planting seeds from a plant comprising the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1, or the nucleic acid construct of the present invention, as defined in the present document, in an area of cultivation of crop plants susceptible to invertebrate pests.

Thus, a tenth object of the present invention is the use of the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1, or the nucleic acid construct of the present invention, as defined in the instant document, wherein the use is for the production of a transgenic plant, wherein the transgenic plant is preferably a monocot, preferably a corn, rice, sugarcane, sorghum, wheat or brachiaria plant.

In a preferred embodiment of the present invention, said use comprises the fact that the transgenic plant is resistant to invertebrate pests.

An eleventh object of the present invention is the use of the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1, or the nucleic acid construct of the present invention, as defined in the present document, wherein the use is for controlling invertebrate pests, preferably insects.

Any of the objects or their preferred embodiments, as described above, can serve as a basis for composing other objects and their preferred embodiments, even if such relationships have not been explicitly described.

The inventors of the present invention have disclosed that, by means of the codon-optimized cry1 Da nucleic acid molecule, as defined in the present document, transgenic maize plants resistant to crop pests were obtained, including pests already resistant to the transgenic maize plants of the state of the art.

### DETAILED DESCRIPTION OF THE INVENTION

### Definitions

Unless otherwise defined, all terms used in the art, annotations and other scientific terminologies used herein are intended to have the meanings usually understood by those skilled in the art in the field of the present invention. In some instances, terms having the commonly understood meanings are defined in the present document for the purpose of bringing clarity and/or for prompt reference, and inclusion of such definitions in the instant document should not necessarily be interpreted as representing a substantial difference relative to what is usually understood in the state of the art.

The techniques and procedures described or referred to in the present document are generally well understood and employed using conventional methodology by those skilled in the art. As appropriate, processes involving the use of commercially available kits and reagents are generally carried out in accordance with protocols and/or parameters defined by the manufacturer, unless otherwise indicated.

It is worth mentioning that the present invention, where appropriate, is not limited to the methodology, protocols, cell line, genera or animal species, constructs and specific reagents as described, which, obviously, may vary. In addition, the terminology used in the present document is only for the purpose of describing examples of specific embodiments thereof, and is not intended to limit the scope of the present invention.

Throughout the instant document, singular forms “a” and “the” or singular forms of any term or expression, include references to the plural, unless the context clearly dictates otherwise.

Throughout the instant document, the word “comprises”, and any variations thereof such as “comprising” or “comprise” should be interpreted as “open terms”, which may imply the inclusion of additional elements or groups of elements, which were not explicitly mentioned, not having a limitative character.

Throughout the present document, the word “consists”, and any variations such as “consist” or “consisting”, should be interpreted as “closed terms”, and may not imply the inclusion of additional elements or groups of elements that were not explicitly described, having a limitative character.

Throughout the instant document, the exact values or ranges of exact values provided with respect to a particular factor, amount, concentration or particular preference should be interpreted as also providing corresponding values or ranges of approximate values, such as through the expression “about”.

Throughout the instant document, words and expressions such as “preferably”, “particularly”, “for example”, “such as”, “as”, “more particularly” and the like, and variations thereof, must be interpreted as entirely optional characteristics, preferred embodiments or possible non-exhaustive examples, without limiting the scope of the invention.

Throughout the instant document, words and expressions such as “nucleic acids”, “nucleotides” and the like should be interpreted as naturally occurring, synthetic or artificial nucleic acids or nucleotides. They comprise deoxyribonucleotides (DNA) or ribonucleotides (RNA) or any nucleotide analog and polymers or hybrids thereof in sense or antisense configuration, being single-stranded or double-stranded. Unless otherwise stated, a specific nucleic acid sequence also implicitly encompasses conservatively modified variants thereof (for example, degenerate codon substitutions) and complementary sequences, as well as the sequences explicitly indicated. The term “nucleic acid” is used interchangeably in the present document with terms “gene”, “cDNA”, “mRNA”, “oligonucleotide”, “nucleic acid molecule” or “primer”.

The expressions “nucleic acid molecule”, “nucleic acid sequence” and the like refer to a polymer of single-stranded or double-stranded DNA or RNA bases, read from the 5′ to the 3′ end. It includes chromosomal DNA, self-replicating plasmid, infectious DNA or RNA polymers that play a mainly structural role, among others. They also refer to a consecutive list of abbreviations, letters, characters or words representing nucleotides or genes, as usually used in the technical field of the present invention.

Throughout this document, the expression “codon-optimized”, when referring to molecules or sequences of the present invention, should be interpreted as the molecule or sequence of nucleotides that has undergone a process to adapt its nucleotide constitution (content C+G and A+T) to the constitution of the host or recipient organism, so that it could more efficiently express a heterologous protein. Codon optimization processes are known to a person skilled in the art.

Throughout the instant document, words and expressions such as “sequence similarity”, “identity” and the like, with respect to another sequence, should be interpreted as the percentage of nucleotides in the sequence that is identical to the nucleotides in another sequence, after alignment of sequences and the introduction of gaps, if necessary, to achieve the maximum percentage of sequence identity. According to the present invention, the expression “at least 70% similarity” is defined as 70 to 100% similarity or identity. Preferably, percentage of similarity is at least 75%, at least 80%, at least 85%, at least 90%, at least 95% or 100%.

Throughout the instant document, the expression “nucleic acid construct” should be interpreted as a single or double stranded, linear or circular DNA construct, which is capable of causing the expression of the protein of interest. Typically, it comprises a promoter sequence and a coding sequence. Yet, typically, constructs may also comprise a 3′ UTR region. Such constructs may comprise other regulatory or signaling sequences known to a person skilled in the art. Preferably, the construct of the present invention comprises the nucleic acid sequence defined as SEQ ID NO: 10.

Throughout this document, words and expressions such as “promoter”, “promoter sequence” and the like should be interpreted as a DNA sequence that, once operatively linked to a nucleotide sequence of interest, is able to control transcription of the nucleotide sequence of interest into RNA. A promoter is located 5′ (or upstream) of the site of initiation of transcription of a nucleotide sequence of interest whose mRNA transcription it controls and provides a site for specific binding of RNA polymerase and other transcription factors for transcription to start. It may include other regulatory sequences known to a person skilled in the art. According to the present invention, the promoter can be heterologous or homologous to the respective cell or host. A nucleic acid sequence is “heterologous” to an organism or a second nucleic acid sequence if it originates from a different species or, if from the same species, it is modified from its original form.

Several promoters are suitable for carrying out the present invention. Suitable non-exhaustive promoters are, for example, promoters 19S and 35S from CaMV cauliflower mosaic virus and FMV scrofularia mosaic virus, either duplicated or not, Arabidopsis and maize ubiquitin (ubi) promoters, nopaline synthase (nos) and octopine synthase (ocs) promoters, the light-inducible promoter of the small subunit of ribulose 1,5-bisphosphate carboxylase (ssRUBISCO), among others. Preferably, the promoter sequence is maize ubiquitin (ubi) promoter sequence, defined as SEQ ID NO: 4, and the duplicated CaMV 35S gene promoter sequence of cauliflower mosaic virus, defined as SEQ ID NO: 6.

Throughout this document, the expression “3′ UTR terminator sequence” should be interpreted as the 3′ terminator sequence of the untranslated region, spanning from the termination codon. Several 3′ UTR terminator sequences are suitable for carrying out the present invention. Suitable, non-exhaustive 3′ UTR terminator sequences are, for example, nopaline synthase (nos) terminator sequence from Agrobacterium, Tvsp sequence of the gene encoding soybean vegetative storage protein, the 3′ region of 35S CaMV and potato pinII proteinase inhibitor gene. Preferably, the 3′ UTR terminator sequence is nopaline synthase (nos) terminator sequence from Agrobacterium, defined as SEQ ID NO: 5, and the Tvsp sequence of the gene encoding the soybean vegetative storage protein, defined as SEQ ID NO: 9.

Throughout the instant document, words and expressions such as “selection gene”, “selectable marker”, “selectable marker gene (SMG)” and the like should be interpreted as genes that produce a product, which serves to select or differentiate the cell or tissue expressing it from other cells or tissues that do not express it. Several selectable genes are suitable for carrying out the present invention. Suitable non-exhaustive selectable genes are, for example, GUS (R-glucuronidase coding sequence), GFP (green fluorescent protein coding sequence), LUX (luciferase coding gene), antibiotic resistance marker genes (as, for example, transposons Tns (bla), Tn5 (nptII), TN7 (dhfr), penicillin, kanamycin, neomycin, methotrexate, tetracycline, etc.) or herbicide tolerance genes (such as the that of modified enzyme 5-enolpyruvyl-3-phosphoshikimate synthase (EPSPS), phosphinothricin acetyl transferase gene (bar) from Streptomyces hygroscopicus, defined as SEQ ID NO: 8, pat gene, isolated from Streptomyces viridochromogenes, among others). The selectable gene according to the present invention is operably linked to at least one promoter sequence and at least one terminator sequence. Preferably, the promoter sequence is that of CaMV 35S gene duplicated from cauliflower mosaic virus and the terminator sequence is Tvsp sequence of the gene that codes for soybean vegetative storage protein. However, other suitable promoter and terminator sequences can also be used, such as, for example, the sequences listed in the instant document.

Throughout the present document, the expression “other regulatory sequences” refers to, for example, to enhancers and other expression control elements (for example, polyadenylation signals), which may be located upstream (5′ UTR region) or downstream (3′ UTR region), or even inside or between other nucleotide sequences described in the invention. Preferably, the regulatory sequence is the nucleic acid sequence of the Tobacco etch virus (tev) translational enhancer region, being defined as SEQ ID NO: 7. Such regulatory sequences are described, for example, in Goeddel, Gene Expression Technology: Methods in Enzymology 185, Academic Press, San Diego, Calif. (1990), and in Gruber & Crosby, in: Methods in Plant Molecular Biology and Biotechnology, eds. Glick & Thompson, Chapter 7, 89-108, CRC Press; Boca Raton, Fla., included herein for reference. Regulatory sequences include those that direct the constitutive expression of a nucleotide sequence in many types of host cells and those that direct the direct expression of the nucleotide sequence only in certain host cells or under certain conditions.

Throughout the present document, the term “transformation” and the like should be interpreted as a process for introducing heterologous DNA into a cell, plant tissue or plant. It can take place under natural or artificial conditions, such as using several methods well known in the art, in a prokaryotic or eukaryotic host cell. The method is usually selected based on the host cell to be transformed and may include, but is not limited to, viral infection, electroporation, lipofection, particle bombardment (bioballistics) and Agrobacterium-mediated methods.

One of the embodiments of the present invention relates to a cell transformation method. Any cell transformation method is included in the scope of the present invention and is not of particular relevance for achieving the embodiments of the invention, as long as it includes introducing into said cell, by means known to a person skilled in the art, the codon-optimized cry1 Da nucleic acid molecule comprising a nucleic acid sequence having at least 70% similarity with the sequence defined as SEQ ID NO: 1, preferably with at least 90% similarity with the sequence of SEQ ID NO: 1, more preferably being SEQ ID NO: 1 or the nucleic acid construct of the present invention, as defined herein. Preferably, the nucleic acid molecule integrates into the cell genome.

Throughout the present document, the term “transgene” should be interpreted as any nucleic acid sequence that is introduced into a cell through experimental manipulations, being integrated into the genome or not. A transgene can be an “endogenous DNA sequence”, or an “exogenous DNA sequence” (i.e., “heterologous”). The term “endogenous DNA sequence” refers to a nucleotide sequence that is naturally found in the cell into which it is introduced. The term “exogenous DNA sequence” refers to a nucleotide sequence that is not naturally found in the cell into which it is introduced. The term “transgenic” in reference to a transformed organism, means an organism transformed with a recombinant DNA molecule that preferably comprises a suitable promoter operably linked to a DNA sequence of interest.

Throughout the present document, the term “vector” should be interpreted as a construct containing a DNA sequence that is operably linked to one or more suitable control sequences capable of leading to the expression of said DNA sequence in a suitable host. Such control sequences include a promoter to perform transcription, an optional operator sequence for controlling such transcription, a sequence coding for the suitable mRNA binding sites to the ribosome, and sequences that control the end of transcription and translation, for example.

Several vectors are suitable for carrying out the present invention. Vectors are, for example, phages, viruses such as SV40, CMV, baculovirus, adenovirus, transposons, IS elements, plasmids, phagemids, cosmids, linear or circular DNA. These vectors can be replicated autonomously in the host organism or be replicated by the chromosome. The vector can also be a plasmid. According to the present document, the terms “plasmid” and “vector” are sometimes used interchangeably. Preferably, the vector according to the present invention comprises the codon-optimized cry1Da nucleic acid molecule, comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct of the, as defined herein.

Throughout the instant document, the expressions “host cell”, “host organism” and the like should be interpreted as being the specific host organism or the specific target cell, but also as being the descendants or potential descendants of those organisms or cells. Since due to mutation or environmental effects certain modifications may appear in successive generations, these descendants need not necessarily be identical to the parental cell. However, they are still included in the scope of protection of the present invention. According to the present invention, host cells can be prokaryotic or eukaryotic. Preferably, the host cell according to the present invention is a plant host cell. Preferably, it comprises the codon-optimized cry1 Da nucleic acid molecule, comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct or the vector of the present invention, as defined herein.

Throughout the present document, words and expressions such as “transgenic plant cell”, “transgenic plant” and the like should be interpreted as cells or plants having and preferably express through experimental manipulations a transgene, and also refer to the progeny of a transgenic plant and subsequent generations of plants, as above.

Throughout the present document, the term “plant” and the like should be interpreted as being the plant organism in whole or in part. “Part” in this context means plant cells and tissues, organs and parts of plants in all their manifestations, such as seeds, leaves, anthers, fibers, tubers, roots, root hair, stems, embryos, calluses, cotyledons, petioles, collected material, plant tissue, reproductive tissue and cell cultures. The transgenic plants according to the present invention can be generated and self-fertilized or crossed with other individuals in order to obtain additional transgenic plants. Transgenic plants can also be obtained by vegetative propagation of transgenic plant cells.

Any transformed plant obtained according to the invention can be used in a conventional breeding scheme or in plant propagation in vitro to produce more transformed plants having the same traits and/or can be used to introduce the same trait into other varieties of the same species or related species. These plants are also part of the invention. Seeds obtained from genetically transformed plants also contain the same trait and are part of the invention. The present invention is applicable to any plant and culture that can be transformed with any of the transformation methods known to those skilled in the art. The plants according to the present invention can be monocotyledonous or dicotyledonous plants. Preferred monocotyledonous plants include, but are not limited to maize, rice, sugar cane, sorghum, wheat or brachiaria plants, more preferably maize. Still preferably still, the plants according to the present invention are transgenic plants resistant to crop pests.

One of the embodiments of the present invention relates to a method of producing a transgenic plant. Any method of producing a transgenic plant is included in the scope of the present invention, and is not of particular relevance for obtaining the embodiments of the invention. Preferably, the method comprises transforming a plant cell with the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct of the present invention, as defined herein. Preferably, the method further comprises selecting a plant cell transformed with the codon-optimized cry1 Da nucleic acid molecule comprising a nucleic acid sequence having at least 70% similarity with the sequence of SEQ ID NO: 1, preferably with at least 90% similarity with the sequence of SEQ ID NO: 1, more preferably being SEQ ID NO: 1 or the nucleic acid construct or the vector of the present invention, as defined herein. Preferably, the method further comprises regenerating a transgenic plant from said plant cell.

Throughout the present document, words and expressions such as “pest”, “crop pests” and the like should be interpreted as invertebrate pests, which include, but are not limited to insects, fungi, bacteria, nematodes, mites, ticks and the like. In particular, insects include those selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, among others. Coleoptera, Lepidoptera and Diptera are preferred.

In particular, the order Lepidoptera includes, without limitation, families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, Crambidae and Tineidae, more particularly Spodoptera sp., particularly Spodoptera frugiperda (Noctuidae) and Diatrea sp., particularly Diatrea saccharalis (Crambidae).

One of the embodiments of the present invention relates to a method of controlling invertebrate pests in crop plants. Any method of controlling invertebrate pests in crop plants is included in the scope of the present invention, not being of particular relevance for achieving the embodiments of the invention, as long as the crop plants according to the present invention comprise the codon-optimized cry1 Da nucleic acid molecule comprising a nucleic acid sequence having at least 70% similarity with the sequence defined as SEQ ID NO: 1, preferably with at least 90% similarity with the sequence of SEQ ID NO: 1, more preferably being SEQ ID NO: 1, or the nucleic acid construct of the present invention, as defined in the present document, wherein the method preferably comprises planting seeds from a plant comprising the nucleic acid sequence having at least 70% similarity with the sequence of SEQ ID NO: 1, preferably with at least 90% similarity with the sequence of SEQ ID NO: 1, more preferably being SEQ ID NO: 1, or the nucleic acid construct of the present invention, as defined in the present document, in an area of cultivation of crop plants susceptible to invertebrate pests.

Throughout the present document, all titles and subtitles are used for convenience only and should not be construed as limitations on the present invention.

### EXAMPLES

### Example 1—Selection of Cry1Da Gene

A search was carried out in B. thuringiensis germplasm bank belonging to the Biological Control Laboratory in order to detect strains capable of controlling the development of Spodoptera frugiperda. Through PCR experiments using specific primers for cry1 Da gene (SEQ ID NO: 18 and SEQ ID NO: 19) (Cerón et al., 1994) strains containing the 290 bp fragment were found. These fragments were sequenced and compared with the NCBI database. Primers representing the 5′ and 3′ border regions of cry1 Da gene based on sequences found on NCBI (SEQ ID NO: 20 and SEQ ID NO: 21) were used to isolate the full-length gene from strain Bt 1132C using high fidelity taq polymerase. The amplified fragment representing full-length cry1 Da gene was cloned into the pGEM vector (Promega) by means of a reaction to add adenine and used to sequence both strands using internal primers. Sequencing was performed using three clones obtained from independent amplifications.

Definition of the sequence for synthesizing the Bt gene present in the gene construct codes for a 625-amino acid protein corresponding to cry1 Da protein active site and was based on three aspects: (i) Presence of C-terminal domain (Endotoxin C), Central domain (Endotoxin M) and N-terminal domain (Endotoxin N); (ii) Size of the protein active core—residues from aa 1 to 625 (Abdul Aziz, H., Wei Hong, L. and Yusoff, K. Comparative study of cry1 D gene expressed in E. coli and Baculovirus expression system/http://www.ncbi.nlm.nih.gov/protein/AFK29089.1); (iii) In silico detection of cry1 Da protoxin hydrolysis sites with trypsin or chymotrypsin enzymes.

### Example 2—Change in Codon of Native Cry1Da Gene

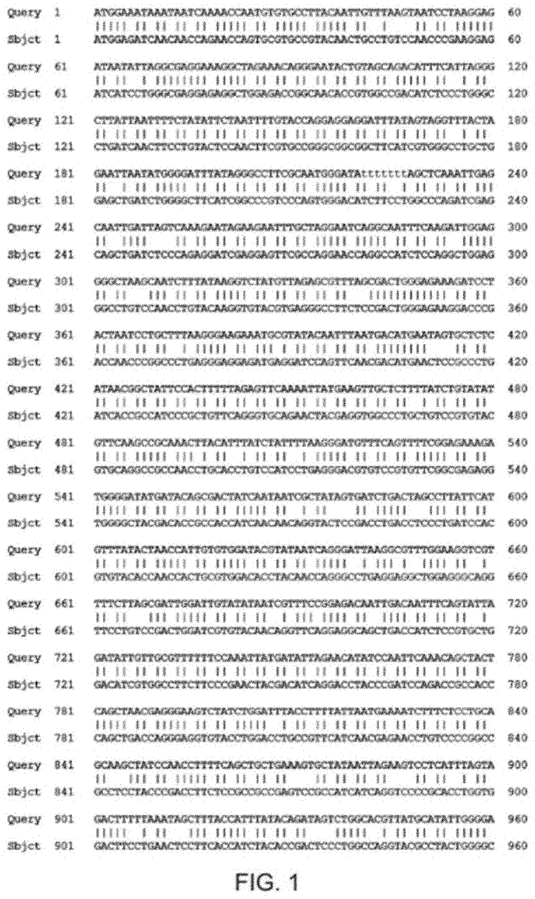

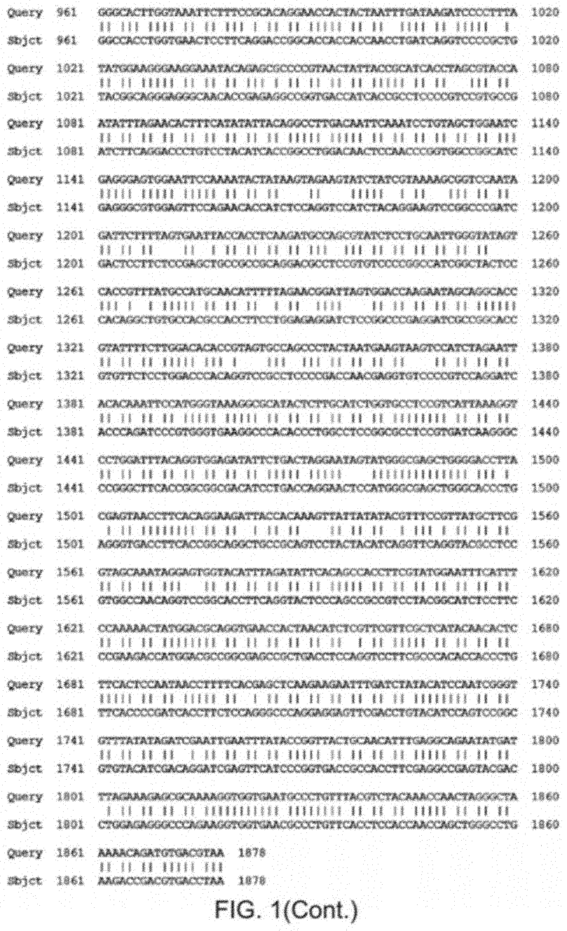

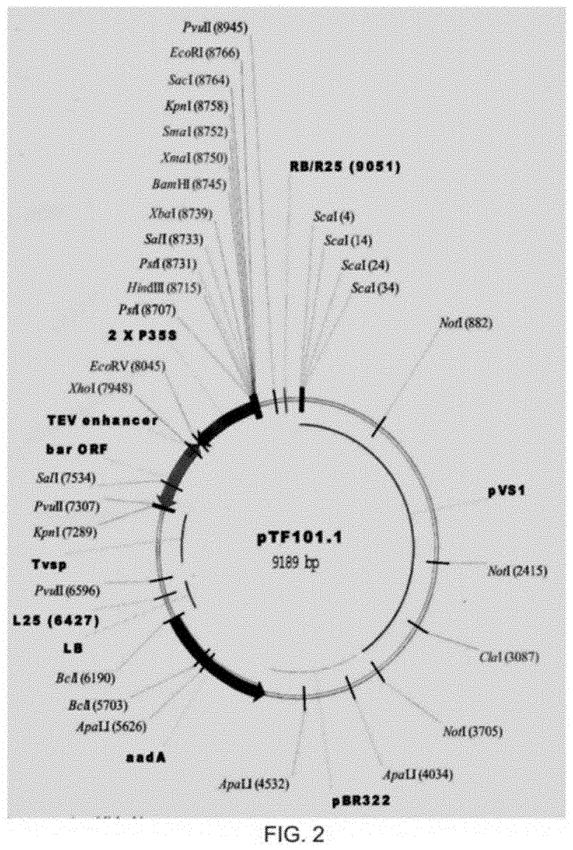

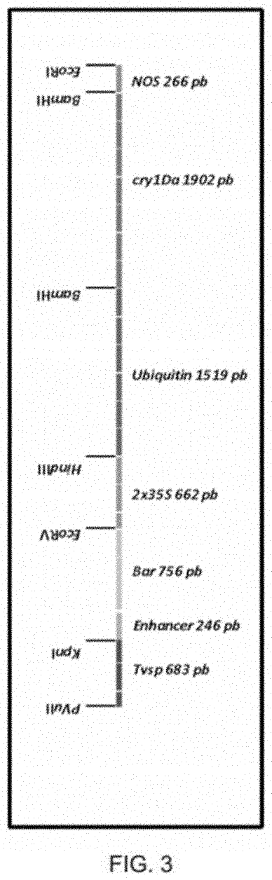

In a second step, the codon of the protein active core was modified—residues from aa 1 to 625/a 1875 bp nucleotide sequence —originally isolated from B. thuringiensis (SEQ ID NO: 2) so as to become compatible with the codon found in maize. cry1Da gene codon modification was made using software Optimizer (http://genomes.urv.es/OPTIMIZER/Form.php) (Puigbo, 2007) and the sequence sent digitally for commercial synthesis. The sequence initially isolated from strain BT 1132C had a CG content of 37.91% and was optimized for a 63.8% CG content (SEQ ID NO: 1) (FIG. 1).

The synthesized fragment was cloned into plasmid pUC19 with restriction enzyme sites compatible for cloning into binary vector pTF101. The synthesized sequence was confirmed by sequencing using standard techniques.

### Example 3—Gene Construct

Cloning of cry1 Da gene 63.8% CG was performed in binary vector pTF101 (Paz et al., 2004) containing maize ubiquitin gene promoter (ubi) and nopaline synthase gene terminator (nos). Plasmids pTF101 and pUCcry1 Da were cleaved with enzymes EcoRI and HindIII. Then, a ligation reaction was performed between the binary vector pTF101 and UBI::cry1 Da::NOS gene. Cleavage and ligation carried out using restriction enzymes and T4 ligase, respectively, were performed according to the manufacturer's instructions (Invitrogen/USA). Result of this cloning was transformed via electroporation (BioRad/MicroPulser) into E. coli HB101 using a spectinomycin selection marker and some colonies of this bacterium were analyzed to check for the presence of UBI::cry1 Da::NOS gene. Cloning and direction were confirmed by PCR and sequencing using the primers described in Table 1, which amplify the fragment referring to the Ubiquitin promoter and cleavage with enzymes HindIII and BamHI. For sequencing, the commercial kit BigDye Terminator v3.1 (Applied Biosystems) was used. Plasmid DNA from two bacterial colonies containing the gene construct was sequenced and compared with the sequence of interest and they were found to be identical.

Once cloning of UBI::cry1 Da::NOS gene into plasmid pTF101 was confirmed, this gene construct was transferred to Agrobacterium tumefaciens EHA101 using the electroporation methodology (BioRad/MicroPulser). The same procedure as above was performed to demonstrate the presence of the binary vector containing the cry1 Da gene in A. tumefaciens. Plasmid DNA was isolated from A. tumefaciens colonies, amplified with primers to detect the bar gene in Table 1 (selection gene present in the binary vector). Plasmid DNA was also cleaved with restriction enzymes and the size of the bands seen on agarose gel was as expected, thus confirming the presence of the gene of interest in A. tumefaciens. All PCR fragment amplification reactions were carried out under the following conditions: 94° C. for 2 min; 35 cycles at 94° C. for 30 sec; 55° C. for 30 sec; 72° C. for 30 sec; one cycle at 72° C. for 5 min. Results were viewed on a 1% agarose gel stained with GelRed (Biotium). Photos were documented in a digital image capture system.

Agrobacterium tumefaciens EHA 101 containing the gene constructs of interest (UBI::cry1 Da::NOST and 35S::bar::35T) was used for genetically transforming maize.

TABLE 1
SEQUENCES OF PRIMERS USED AND SIZE OF THEIR
RESPECTIVE AMPLICONS

| | | | SEQ ID |
|---|---|---|---|
| Primer | 5′-3′ sequence | Amplicon | NO: |
| U4 | ACCGGCTAGAGCCATCCCAG | ≈600 pb | SEQ ID |
| | | | NO: 12 |
| U1 | ACCAACCAGCGAACCAG | | SEQ ID |
| | | | NO: 13 |
| BAR-F1 | AGAAACCCACGTCATGCC | 413 pb | SEQ ID |
| | | | NO: 14 |
| BAR-R1 | TGCACCATCGTCAACCAC | | SEQ ID |
| | | | NO: 15 |
| Ubi-F | TTGATGTGGGTTTTACTGATGC | 589 pb | SEQ ID |
| | | | NO: 16 |
| Cry1Da-R | CACCTTGTACAGGTTGGACA | | SEQ ID |
| | | | NO: 17 |

### Example 4—Genetic Transformation of Immature HiII Maize Embryos Via Agrobacterium Tumefaciens

The genotype used in this transformation protocol is Hill maize (Armstrong et al., 1991), according to the protocol by Frame et al. (2002), with minor modifications. Briefly, for transformation of this genotype, immature embryos of between 1.8-2.0 mm in length (10-12 days after pollination) were collected. Spikes used to collect the embryos were dipped in a 1:1 solution of commercial bleach (2.5% sodium hypochlorite) and distilled H₂O with 1 to 2 drops of Tween 20, for 20 minutes. Then, they were rinsed with sterile distilled water for 5 minutes, twice.

Immature embryos were collected with the aid of a spatula from a superficial cut of the grains. To transfer the gene construct to maize, Agrobacterium tumefaciens EHA101 was used. From a stock culture of A. tumefaciens containing the gene construct of interest kept in glycerol at −80° C. a streak was made in YEP medium (5 g·L⁻¹ yeast extract; 10 g·L⁻¹ peptone; 5 g·L⁻¹ NaCl; 15 g·L⁻¹ bacto agar) containing the necessary antibiotics (100 mg·L⁻¹ spectinomycin and 50 mg·L⁻¹ kanamycin) and the plate was incubated for 2 to 3 days at 28° C., parent plate). For genetic transformation, Agrobacterium was streaked using a colony isolated from the parent plate in YEP medium containing the necessary antibiotics. The plate was incubated for 2 to 5 days at 19° C. Then, Agrobacterium was resuspended in infection medium (4.0 gL g·L⁻¹ N6 salts; 68.4 g·L⁻¹ sucrose; 36.0 g·L⁻¹ glucose; 0.7 g·L⁻¹ proline; 1.5 mg·L⁻¹ 2,4-D; 1.0 mL·L⁻¹ N6 vitamins (1000×=1.0 g·L⁻¹ thiamine HCl; 0.5 g·L⁻¹ pyridoxine HCl; 0.5 g·L⁻¹ nicotinic acid); pH 5.2) supplemented with 100 μM acetosyringone until OD550=0.3-0.4 was reached and incubated in a shaker at ≣150 rpm, 23° C. for 2 hours.

For infection of immature maize embryos, 50 to 100 embryos were collected in 1 mL of infection medium plus acetosyringone. After collection, the embryos were rinsed twice, 1 mL of the bacterial culture was added, and the suspension incubated for five minutes at 23° C. After infection, the embryos were transferred to the surface of co-culture medium (4.0 g·L⁻¹ N6 salts; 1.5 mg·L⁻¹ 2.4-D; 30.0 g·L⁻¹ sucrose; 0.7 g·L⁻¹ proline; 1.0 mL·L⁻¹ N6 vitamins (1000×); 0.85 mg·L⁻¹ AgNO₃; 100 μM acetosyringone; 300 mg·L⁻¹ L-cysteine; 3.0 g·L⁻¹ phytagel; pH 5.8) with the scutellum facing upwards. Plates were incubated in the dark at 20° C. for 3 to 5 days. After co-cultivation the embryos were transferred to the resting medium (4.0 g·L⁻¹ N6 salts; 1.5 mg·L⁻¹ 2.4-D; 30.0 g·L⁻¹ sucrose; 0.5 g·L⁻¹ MES; 0.7 g·L⁻¹ proline; 1.0 mL·L⁻¹ vitamins N6 (1000×); 0.85 mg·L⁻¹ AgNO₃; 100 mg·L⁻¹ Tioxin; 3.0 g·L⁻¹ phytagel; pH 5.8) at 28° C. (dark) for 7 to 15 days. Then, the embryos were transferred to the selection medium (4.0 g·L⁻¹ N6 salts; 1.5 mg·L⁻¹ 2.4-D; 30.0 g·L⁻¹ sucrose; 0.5 g·L⁻¹ MES; 0.7 g·L⁻¹ proline; 1.0 mL·L⁻¹ vitamins N6 (1000×); 0.85 mg·L⁻¹ AgNO₃; 100 mg·L⁻¹ Tioxin; 1.5 and 3.0 mg/L bialaphos; 3.0 g·L⁻¹ phytagel; pH 5.8) (25 embryos/plate). Subcultures of these embryos in selective media are carried out every 15 days to select vigorously growing callus.

Selected calluses were transferred to regeneration medium (4.62 g·L⁻¹ MS salts; 60.0 g·L⁻¹ sucrose; 100 mg·L⁻¹ myo-inositol; 1.0 mL·L⁻¹ MS vitamins (1000×); 1.5 mg/L bialaphos; 4.0 g·L⁻¹ phytagel; pH 5.8) and incubated at 26±2° C. (dark) for 15 to 21 days. Ready for germination calluses having a dry appearance and opaque white color were transferred to the germination medium (4.62 g·L⁻¹ MS salts; 30.0 g·L⁻¹ sucrose; 100 mg·L⁻¹ myo-inositol; 1.0 mL·L⁻¹ MS vitamins (1000×=0.5 gL⁻¹ thiamine HCl; 0.5 g·L⁻¹ pyridoxine HCl; 0.05 g·L⁻¹ nicotinic acid); 3.0 g·L⁻¹ phytagel; pH 5.8) (12 calluses per plate), 25° C., 80-100 ρE/m²/sec of light intensity, 16 hour-photoperiod). Seedlings with leaves and roots were transferred to the greenhouse within 14 to 20 days.

When the roots were well developed and the leaf structures were about 5 cm long, the seedlings were transplanted into pots in a greenhouse containing a commercial mixture of soil and organic matter (⅔ soil and ⅓ organic matter (TDP 30/15).

### Example 5—Generation of Cry1Da Gene-Containing Transgenic Maize Events

Maize events containing the codon-optimized cry1 Da nucleic acid sequence of the present invention (SEQ ID NO: 1) were generated, according to Table 2 below. Event ME240913 (Event 01) was generated by transforming the Hill hybrid, which was introgressed into the L3 tropical strain using molecular marker-assisted selection.

TABLE 2

| | Number of seeds produced |
|---|---|
| Events Produced | in the 1^{st} generation |
| ME240913 - Event 01 | 101 |
| ME240913 - Event 04 | 03 |
| ME260913 - Event 02 | 33 |

### Example 6—Bioassay

In order to evaluate the susceptibility of transgenic maize expressing Cry1 Da protein (encoded by the codon-optimized nucleic acid molecule of the present invention), bioassays were performed in the laboratory.

The tests were carried out as follows: Spodoptera frugiperda neonate caterpillars were inoculated into leaves of cry1 Da transgenic maize plants kept in a greenhouse and into non-transgenic isoline (5 caterpillars per container) at stages V7 and V8. After infestation, the containers were closed and damage assessment was made after 05 days. In each case, the experimental design consisted of a treatment group (event ME240913 (Event 1) of transgenic, cry1 Da construct-containing maize) and a control group (non-transgenic maize).

The parameters evaluated were: injury score using the scale proposed by Carvalho, 1970 (0: plant with undamaged leaves; 1: plant with shaved leaves; 2: plant with perforated leaves; 3: plant with torn leaves; 4: plant showing damage to the cartridge, and 5: plant showing a destroyed cartridge); caterpillar survival (the number of surviving caterpillars in each pot was counted); and caterpillar biomass (using a precision scale of four decimal points).

### Example 7—Bioassays to Control Spodoptera Frugiperda and Diatrea Saccharalis Using the Generated Transgenic Maize Events

Spodoptera frugiperda assays: first, event ME240913 (Event 1) was tested for S. frugiperda control. Event seeds were germinated in a greenhouse and when the plants reached the stage between the 10 and 12 leaf stage (end of vegetative stage), the two youngest leaves of each plant were used in Spodoptera frugiperda bioassays. Three repetitions were performed, five caterpillars per repetition. Hill and L3 maize leaves were used as a negative control (caterpillars grow normally) and Viptera maize leaves as a positive control (caterpillars cannot grow). In this first test, it was found that event ME240913 (Event 1) had a good ability to control the caterpillar development, reaching 100% mortality (Table 3).

TABLE 3
EVALUATION OF TRANSGENIC MAIZE EVENTS COMPARED TO
SPODOPTERA FRUGIPERDA CONTROL (BIOASSAY 1)

| | Live | | |
|---|---|---|---|
| | caterpillars | | Total weight of |
| | (After 05 | Dead | live caterpillars |
| Event/Repetition | days) | caterpillars | (mg) |
| ME240913 - Event 1 | 0 | 5 | 0 |
| (cry1Da)/1 | | | |
| ME240913 - Event 1 | 0 | 5 | 0 |
| (cry1Da)/2 | | | |
| ME240913 - Event 1 | 0 | 5 | 0 |
| (cry1Da)/3 | | | |
| Viptera Maize Control+/1 | 0 | 5 | 0 |
| Viptera Maize Control+/2 | 0 | 5 | 0 |
| Viptera Maize Control+/3 | 0 | 5 | 0 |
| Hill Maize Control−/1 | 5 | 0 | 70.3 |
| Hill Maize Control−/2 | 4 | 1 | 77.3 |
| Hill Maize Control−/3 | 5 | 0 | 68.7 |
| L3 Maize Control−/1 | 4 | 1 | 39.5 |
| L3 Maize Control−/2 | 5 | 0 | 84.4 |
| L3 Maize Control−/3 | 5 | 0 | 61.6 |

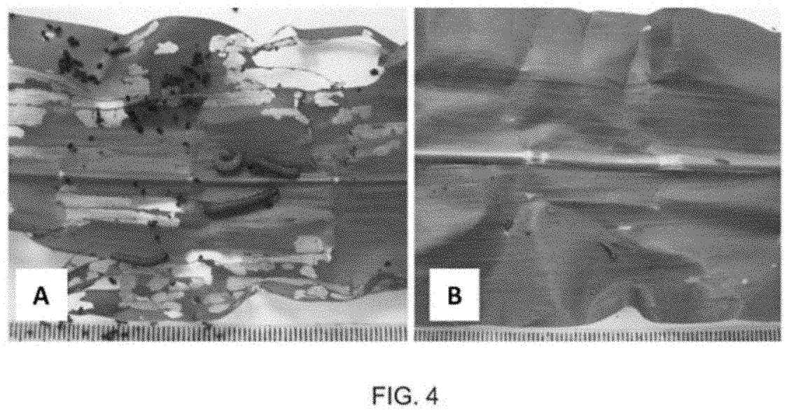

The bioassay with this event was repeated but this time four repetitions were made with 20 caterpillars per repetition, and the results confirmed that this event had the ability to control the development of S. frugiperda (FIG. 4 representative of feeding bioassays with Spodoptera frugiperda in non-transgenic maize and transgenic maize of the present invention). At a different time, another bioassay was carried out to test two more of the generated cry1Da events: ME260913—Event 2 (cry Da) and ME240913—Event 4 (cry1 Da) (Table 4). The two generated events were also able to efficiently control the development of S. fugiperda.

TABLE 4
EVALUATION OF TRANSGENIC MAIZE EVENTS COMPARED TO
SPODOPTERA FRUGIPERDA CONTROL (BIOASSAY 2)

| | | | Total weight of |
|---|---|---|---|
| | Live | Dead | live caterpillars |
| Event | caterpillars | caterpillars | (mg) |
| ME240913 - Event 1 | 0 | 20 | 0 |
| (cry1Da)/1 | | | |
| ME240913 - Event 1 | 0 | 20 | 0 |
| (cry1Da)/2 | | | |
| ME240913 - Event 1 | 0 | 20 | 0 |
| (cry1Da)/3 | | | |
| ME240913 - Event 1 | 0 | 20 | 0 |
| (cry1Da)/4 | | | |
| ME240913 - Event 4 | 0 | 20 | 0 |
| (cry1Da)/1 | | | |
| ME240913 - Event 4 | 0 | 20 | 0 |
| (cry1Da)/2 | | | |
| ME240913 - Event 4 | 0 | 20 | 0 |
| (cry1Da)/3 | | | |
| ME260913 - Event 2 | 1 | 19 | 0.7 |
| (cry1Da)/1 | | | |
| ME261913 - Event 2 | 0 | 20 | 0 |
| (cry1Da)/2 | | | |
| ME260913 - Event 2 | 0 | 20 | 0 |
| (cry1Da)/3 | | | |
| Hill/1 | 18 | 02 | 176.7 |
| Hill/2 | 18 | 02 | 131.7 |
| Hill/3 | 15 | 05 | 137.0 |
| Hill/4 | 17 | 03 | 212.7 |
| L3/1 | 19 | 01 | 131.9 |
| L3/2 | 19 | 01 | 132.5 |
| L3/3 | 19 | 01 | 133.3 |
| L3/4 | 18 | 02 | 115.6 |

Assays using Diatrea saccharalis: the same events tested for S. frugiperda were also tested for Diatraea saccharalis. Five one day-old caterpillars were placed on leaves of transgenic maize events and controls. Event ME240913 (Event 1) was also found to be able to control the development of D. saccharais. Within this period of time, the caterpillars did not die, but were unable to grow (Table 5).

TABLE 5
EVALUATION OF TRANSGENIC MAIZE EVENTS COMPARED
TO DIATRAEA SACCHARALIS CONTROL (BIOASSAY 1)

| | Live | | |
|---|---|---|---|
| | caterpillars | | Total weight of |
| | (After 05 | Dead | live caterpillars |
| Event | days) | caterpillars | (mg) |
| ME240913 - Event 1 | 05 | 0 | >0.1 |
| (cry1Da)/1 | | | |
| ME240913 - Event 1 | 05 | 0 | >0.1 |
| (cry1Da)/2 | | | |
| ME240913 - Event 1 | 04 | 1 | 0.2 |
| (cry1Da)/3 | | | |
| Viptera Maize Control+/1 | 05 | 0 | >0.1 |
| Viptera Maize Control+/2 | 04 | 1 | >0.1 |
| Viptera Maize Control+/3 | 03 | 2 | 0.2 |
| Hill Maize Control−/1 | 05 | 0 | 6.7 |
| Hill Maize Control−/2 | 05 | 0 | 7.6 |
| Hill Maize Control−/3 | 04 | 1 | 5.7 |
| L3 Maize Control−/1 | 05 | 0 | 6.3 |
| L3 Maize Control−/2 | 05 | 0 | 20.5 |
| L3 Maize Control−/3 | 04 | 1 | 4.6 |

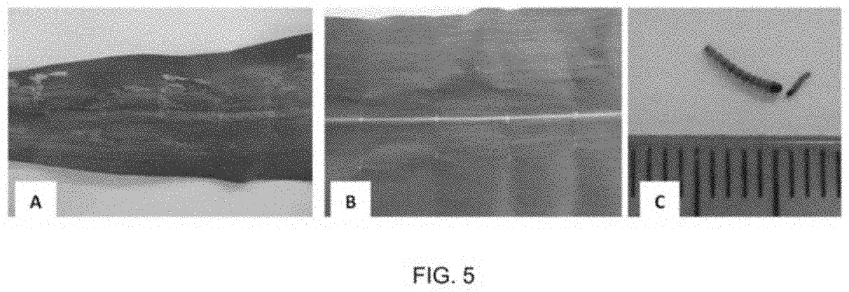

A new bioassay was carried out with this event to confirm toxicity. This time, four repetitions and 20 caterpillars were used in each repetition. The same result as previously obtained was achieved in this second assay (FIG. 5 representative of the feeding bioassays with Diatrea saccharalis in non-transgenic maize and transgenic maize of the present invention). At a different time, another bioassay was carried out to test one more of the generated cry1Da events, ME260913—Event 2 (cry1Da) (table 6). This event was also able to efficiently control the development of D. saccharalis.

TABLE 6
EVALUATION OF TRANSGENIC MAIZE EVENTS COMPARED
TO DIATRAEA SACCHARALIS CONTROL (BIOASSAY 2)

| | Live | | |
|---|---|---|---|
| | caterpillars | | Total weight of |
| | (After 05 | Dead | live caterpillars |
| Event | days) | caterpillars | (mg) |
| ME240913 - Event 1 | 0 | 20 | 0 |
| (cry1Da)/1 | | | |
| ME240913 - Event 1 | 0 | 20 | 0 |
| (cry1Da)/2 | | | |
| ME240913 - Event 1 | 0 | 20 | 0 |
| (cry1Da)/3 | | | |
| ME240913 - Event 1 | 0 | 20 | 0 |
| (cry1Da)/4 | | | |
| ME260913 - Event 2 | 0 | 20 | 0 |
| (cry1Da)/1 | | | |
| ME260913 - Event 2 | 0 | 20 | 0 |
| (cry1Da)/2 | | | |
| ME260913 - Event 2 | 0 | 20 | 0 |
| (cry1Da)/3 | | | |
| Viptera Maize Control+/1 | 04 | 16 | >0.1 |
| Viptera Maize Control+/2 | 02 | 18 | >0.1 |
| Viptera Maize Control+/3 | 04 | 16 | >0.1 |
| Viptera Maize Control+/4 | 0 | 20 | >0.1 |
| Hill Maize Control−/1 | 15 | 05 | 12.0 |
| Hill Maize Control−/2 | 20 | 0 | 21.2 |
| Hill Maize Control−/3 | 18 | 02 | 20.6 |
| Hill Maize Control−/4 | 19 | 01 | 22.4 |
| L3 Maize Control−/1 | 20 | 0 | 22.8 |
| L3 Maize Control−/2 | 20 | 0 | 19.6 |
| L3 Maize Control−/3 | 20 | 0 | 22.8 |
| L3 Maize Control−/4 | 20 | 0 | 24.0 |

### Example 8—Analysis of Cry1Da Gene Expression in Transgenic Maize Events

Cry1 Da gene expression was analyzed using the quantitative polymerase chain reaction (qPCR) assay, according to a standard technique known to a person skilled in the art. Results of this analysis have shown that the events capable of inhibiting the growth of caterpillars express cry1 Da gene efficiently.

### Example 9—Bioassays Using Caterpillars Resistant to Cry1F Gene-Containing Transgenic Maize

Assays were performed to verify the potential of the codon-optimized cry1 Da nucleic acid molecule of the present invention to control cry1F-resistant S. frugiperda.

The experiment was carried out in a greenhouse by planting transgenic maize comprising the codon-optimized cry1 Da nucleic acid molecule of the present invention (ME240913 (Event 1)) and non-transgenic corn (negative control). Neonate caterpillars belonging to two different Spodoptera frugiperda populations were inoculated into maize plants (15 caterpillars per plant) at V7 and V8 stages. After infestation, the pots were isolated with a voil cage and damage assessment was made after 07, 14 and 21 days. The experimental design consisted of 04 treatments, with 05 pots each, containing from 02 to 03 maize plants per pot:

Treatment 1: The transgenic event of the invention ME240913 (Event 01) infested with the Spodoptera frugiperda population resistant to Cry1F protein according to Leite et al., 2016.

Treatment 2: Non-transgenic L3 isogenic line infested with the Cry1F-resistant Spodoptera frugiperda population according to Leite et al 2016.

Treatment 3: The transgenic event of the invention, ME240913 (Event 01), infested with the population of susceptible caterpillars reared and maintained in the entomology laboratory of Embrapa Milho e Sorgo.

Treatment 4: Non-transgenic isogenic line infested with the population of susceptible caterpillars reared and maintained in the entomology laboratory of Embrapa Milho e Sorgo.

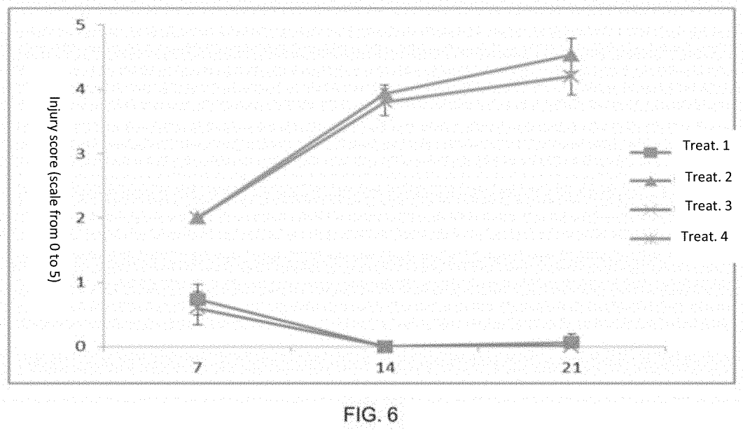

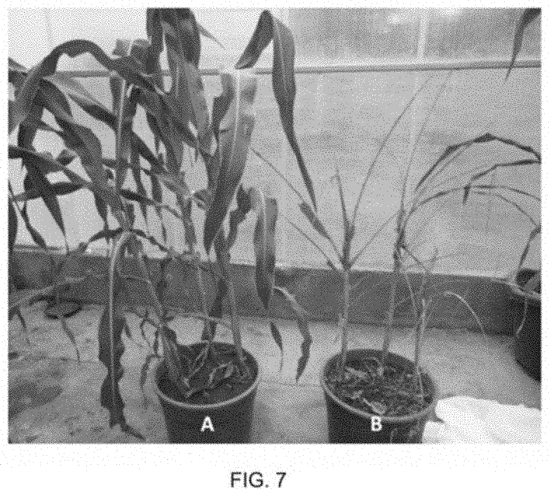

Results have shown that the transgenic plant comprising the codon-optimized cry1 Da nucleic acid molecule of the invention was able to control infestation with Cry1F protein resistant-Spodoptera frugiperda population so effectively as the susceptible population by inhibiting its development (FIG. 6) and protecting the plant from the attack by such a pest (FIG. 7), as observed by the injury score (±IC, P=0.05). Survival percentage of Spodoptera frugiperda, as assessed 21 days after the caterpillars were placed under different treatments, was 0% for treatments 1 and 3, and about 65% and 35% for treatments 2 and 4, respectively. Spodoptera frugiperda biomass, evaluated 21 days after the caterpillars were placed under different treatments, was 0% for treatments 1 and 3, and about 260 mg and 300 mg for treatments 2 and 4, respectively. In both cases, the non-overlapping Cl averages differ from each other (P=0.05).

### BIBLIOGRAPHY

An G, Mitra A, Hong K C, Costa M A, Na K, Thornburg R W et al. (1989) Functional analysis of the 3′ control region of the potato wound-inducible proteinase inhibitor II gene. Plant Cell 1:115-122.
Armstrong C L, Green C E (1985) Establishment and maintenance of friable, embryogenic maize callus and the involvement of L-proline. Planta. 164(2):207-214.
Armstrong C L, Grenn C E, Phillips R L (1991). Development and availability of germplasm with high type II culture formation response. Maize Genet. Coop. Newsl. 65:92-93.
Baum A B, T B Johnson, B C Carlton (1999) Bacillus thuringiensis—Natural and recombinant biopesticide products, p. 189-209. In F. R. Hall & J. J. Menn (eds.), Methods in Biotechnology: Biopesticides: Use and delivery. Totowa, Humana Press, 626p.
Becker D, Kemper E, Schell J, Masterson R (1992) New plant binary vectors with selectable markers located proximal to the left T-DNA border. Plant Mol. Biol. 20:1195-1197.
Bevan (1984). Binary Agrobacterium tumefaciens Vectors for plant transformation. Nucleic Acids Research 12:8711-8721.
Bohorova N E, Luna B, Brito R M, Huerta L D, Hoisington D A (1995) Regeneration potential of tropical, subtropical, midaltitude, and highland maize inbreeds. Maydica. 40:275-281.
Bohorova N, Maciel A M, Brito R M et al. Selection and characterization of mexican strains of Bacillus thuringiensis active against four major lepidopteran maize pests. Entomophaga, Paris, v. 4, p. 153-165, 1996.
Boucias D G, Pedland J C (1998) Principles of insect pathology. Boston. Kluwer Academic Publishers, 537p.
Bravo A, Sarabia S, Lopez L, Ontiveros H, Abarca C, Ortiz A, Ortiz M, Lina L, Villalobos F J, Pena G, Nuhez-Valdez M, Soberon M, Quintero R. (1998) Characterization of cry genes in a Mexican Bacillus thuringiensis strains collection. Appl. Environ. Microbiol. 64: 4965-4972.
Buchanan B, Gruissem W, Jones R (2000) Biochemistry and Molecular Biology of Plants (Rockville: ASPP).
Cambell, C. H., and Gowri, G. (1990) Codon usage in higher plants, green algae, and cyanobacteria. Plant Physiol. 92, 1-11.
Carozzi N B, Kramer V C, Warren G W, Evola S, Koziel M G (1991) Prediction of insectidal activity of Bacillus thuringiensis strains by polymerase chain reaction product profiles. Appl. Environ. Microbiol. 57(11):3057-3061.
Carvalho CHS, Bohorova N E, Bordall P N, Abreu L L, Valicente F H, Bressan W, Paiva E (1994) Type-II callus production and plant regeneration in tropical maize genotypes. Plant Cell Rep. 17:73-76.
Carvalho R P L (1970) Danos, flutuação da populagao, controle e comportamento de Spodoptera frugiperda (J. E. Smith, 1797) e susceptibilidade de diferentes genotipos de milho, em condições de campo. Doctorate Thesis. Escola Superior de Agricultura “Luiz de Queiróz”, Piracicaba. 170p.
Chinault A C, Blakesley V A, Roessler E, Willis D G, Smith C A, Cook R G, Fenwick R G (1986) Characterization of transferable plasmids from Shigella flexneris that confer resistance to trimethoprim, streptomycin and sulfonamides. Plasmids 15:119-131.
Christensen, A. H.; Quail, P. H. Ubiquitin promoter-based vectors for high-levels expression of selectable and/or screenable marker genes in monocotyledonous plants. Transgenic Research, v. 5, p. 213-218, 1996.
Cerón J, Covarrubias L, Quintero R, Ortiz A, Ortiz M Aranda, E Lina L, Bravo A (1994) PCR analysis of the cry1 insecticidal crystal family genes from Bacillus thuringiensis. Appl. Environ. Microbiol. 60: 353-356.
Cerón J, Ortiz A, Quintero R, Guereca I, Bravo A (1995). Specific PCR primers directed to identify cry1 and cry1II genes within Bacillus thuringiensis strain collection. App. Environ. Microbiol. 61(11):3826-3831.
De Block M, De Brower D, Tenning P (1989) Transformation of Brassica napus and Brassica oleracea using Agrobacterium tumefaciens and the expression of the bar and neo genes in the transgenic plants. Plant Physiol. 91:694-701.
Depicker, A.; Stachel, S.; Dhaese, P.; Zambryski, P.; Goodman, H. M. Nopaline synthase: transcript mapping and DNA sequence. J. Mol. Appl. Genet., v. 1, p. 561-573, 1982.
Diehn S H, Chiu W L, De Rocker E J, Green P J (1998) Prematuration polyadenilation at multiple sites within a Bacillus thruringiensis toxin gene-coding region. Plant Physiol. 117:1433-1443.
Duncan D R, Williams M E, Zehr B E, Widholm J M The production of callus capable of plant regeneration from immature embryos of numerous Zea mays genotypes. Planta 1985. 165(3):322-332.
Federici B (2002) Case study: Bt crops. In: Atherton K (Ed) Genetically modified crops: assessing safety (chapter 8). Taylor & Francis, New York.
Frame B R, Shou H, Chikwamba R K, Zhang Z, Xiang C, Fonger T M, Pegg E K, Li B, Nettleton D S, Pei D, Wang K (2002) Agrobacterium tumefaciens-Mediated transformation of maize embryos using a standard binary vector system. Plant Physiol. 129:13-22.
Frame, B; Wang K. 2004. Maize Transformation. In: Transgenic crops of the world—Essential Protocols. Ian S. Curtis (Ed). Kluwer Academic Publishers, Netherlands. pp. 45-61.
Gallie D R, Tanguay R L, Leathers V (1995) The tobacco etch virus 5′leader and poly(A) tail are functionally synergistic regulators of translation. Gene 1:561-573.
Glare T R, M. O'Callaghan (2000) Bacillus thuringiensis: Biology, Ecologyand Safety. JohnWiley & Sons, Ltd., 350p.
Gelvin S B (2003) Agrobacterium-mediated plant transformation: the biology behind the “gene-jockeying” tool. Microbiol. Mol. Biol. Rev. 67 (1)16-37.
Gordon-Kamm W J, Spencer T M, Mangano M L, Adams T R, Daines R J, Start W G, O'Brien J V, Chambers S A, Adams Jr. W R, Willetts N G, Riche T B, Mackey C J, Krueger R W, Kausch A P, Lemaux P G (1990) Transformation of maize cells and regeneration of fertile transgenic plants. Plant Cell 2:603-618.
Gustafsson C, Govindarajan S, Minshull J (2004) Codon bias and heterologous protein expression. Trends Biotechnol. 22, 346-353.
Hajdukiewicz P, Svab Z, Maliga P (1994) the small, versatible pPZP Family of Agrobacterium binary vectors for plant transformation. Plant MOl. Biol. 25:989-994.
Hiei Y, Ohta S, Komari T, Kumasho T (1994) Efficient transformation of rice mediated by Agrobacterium and sequence analysis of boundaries of the T-DNA. Plant J. 6:271-282.
Huang X-Q, Wei Z-M (2004) High-frequency plant regeneration through callus initiation from mature embryos of maize (Zea mays L.). 22(11):793-800.
Ikemura, T1985. Codon usage and tRNA content in unicellular and multicellular organisms. Mol. Biol. Evol. 2: 13-34.
Ishida Y, Hiei Y, Komari T (2007) Agrobacterium-mediated transformation of maize. Nature Protocols 2:1614-1621.
Ishida V, Saito H, Ohta S, Hiei Y, Komari T, Kumashiro T (1996) High efficiency transformation of maize (Zea mays L.) mediated by Agrobacterium tumefaciens. Nature Biotechnol. 6:745-750.
Jarrett P, Burges H D (1982) Effect of bacterial varieties on the susceptibility of the greater wax moth Galleria mellonella to Bacillus thuringiensis and its significance in classification of the bacterium. 31(4):346-352.
Joshi C P (1987) Putative polyadenylation signals in nuclear genes of higher plants: a compilation and analysis. Nucleic Acids Res. 15(23): 9627-9640.
Leite, N. A., Mendes, S. M., Santos-Amaya, O. F., Santos, C. A., Teixeira, T. P., Guedes, R. N., & Pereira, E. J. (2016). Rapid selection and characterization of Cry1F resistance in a Brazilian strain of fall armyworm. Entomologia Experimentalis et Applicata, 158(3), 236-247.
Lessard P A, Kulaveerasingam H, York G M, Strong A, Sinskey A J. 2002. Manipulating gene expression for the metabolic engineering of plants. Metabolic Engineering 4, 67-79.
Liu D (2009). Design of gene constructs for transgenic maize. In: Methods in Molecular Biology: Transgenic Maize. M. Paul Scott (ed.). Humana Press. USA. 526:3-20.
Lupotto E, Conti E, Reali A, Lanzanova C, Baldoni E, Allegri L (2004) Improving in vitro culture and regeneration conditions for Agrobacterium-mediated maize transformation. Maydica 49:221-29.
Mason H S, DeWald D, Mullet J E (1993) Identification of a methyl jasmonate-responsive domain in the soybean vspB promoter. Plant Cell 5:241-251.
Murray, E. E., Lotzer, J., and Eberle, M. (1989) Codon usage in plants. Nucleic Acids Res. 17, 477-498.
Odell J T, Nagy F, Chua N H. (1985) Identification of DNA sequences required for activity of the cauliflower mosaic virus 35S promoter. Nature 6:810-812.
Novac F J, Dolezelova M, Nesticky M, Piovaric A (1983) Somatic embryogenesis and plant regeneration in Zea mays L. Maydica. 23:381-390.
Ohme-Takagi M, Taylor C B, Newman T C, Green P (1993) The effect of sequences with high A U content on mRNA stability in tobacco. Proc. Natl Acad. Sci. USA. 90:11811-11815.
Paz M; Shou H; Guo Z; Zhang Z; Banerjee A; Wang K. Assessment of conditions affecting Agrobacterium-mediated soybean transformation using cotyledonary node explant. Euphytica, 136:167-179 (2004).
Petrillo C P, Carneiro N P, Purcino A A C, Carvalho C H S, Alves J D, Carneiro A A (2008) Otimização dos parãmetros de bombardeamento de particulas para a transformação genética de linhagens brasileiras de milho. Pesq. Agropec. Bras. 43(3):371-378.
Perlak, F. J.; Fuchs, R. L.; Dean, D. A.; Mcpherson, S. L.; Fishholff, D. A. 1991. Modification of the coding sequence enhances plant expression of insect control protein genes. Proceedings of the National Academy of Sciences of the USA, 88:3324-3328.
Phillips R L, Somers D A, Hibberd K A (1988) Cell/Tissue Culture and in vitro manipulation p. 345-387. Corn and corn improvement-agronomy. Monograph no. 18, Madison, 3 ed.
Priolli L M, Silva W J (1989) Somatic embryogenesis and plant regeneration capacity in tropical maize inbreds. Rev. Brasil. Genet. 12:553-566.
Puigbo P., Guzmen E., Romeu A. and Garcia-Vallvé S. 2007. OPTIMIZER: A web server utility that optimize a DNA or Protein sequence. Nucleic Acids Research, 35:W126-W131.
Rapela M A (1985) Organogenesis and somatic embryogenesis in tissue cultures of Argentina maize (Zea mays L.) J. Plant Physiol. 121:119-122.
Santos-Serejo J A, Aguiar-Perecin M L R (2000) Genótipos de milho com alta capacidade para embriogênese somática e regerneragao de plantas obtidos a partir de calos. Sci. Agri. 57(4):717-722.
Schafer W, Gorz A, Gunter K (1987) T-DNA integration and expression in a monocot crop plant after induction of Agrobacterium tumefaciens. Nature 327:529-532.
Shaw, G., and Kamen, R. (1986) A conserved AU sequence from the 3′ untranslated region of GM-CSF mRNA mediates selective mRNA degradation. Cell 46, 659-667.
Thompson C J, Movva N R, Tichard R, Crameri R, Davies J E, Lauwereys M (1987) Characterization of the herbicide-resistance gene bar from Streptomyces hygroscopios. EMBO J. 6:2519-2523.
Tojo, A., Aizawa, K. 1983. Dissolution and degradation of Bacillus thuringiensis δ-endotoxin by gut juice protease of the silkworm Bombyx mori. Appl Environ Microbiol 45: 576-580.
Valicente F H, Barreto M R (2003) Bacillus thuringiensis survey in Brazil: geographical distribution and insecticidal activity against Spodoptera frugiperda (J. E. Smith) (Lepidoptera: Noctuidae). Neotropical Entomology. 32(4):639-644.
Valicente F H, Barreto M R, Vasconcelos M J V, Figueiredo J E F, Paiva E (2000). Identificação através de PCR dos genes Cry1 de cepas de Bacillus thuringiensis Berliner eficientes contra a lagarta do cartucho, Spodoptera frugiperda (J. E. Smith) (Lepidoptera: Noctuidae). Anais da Sociedade Entomológica do Brasil. 29(1):147-153.
Vasil I K (1987) Developing cell and tissue culture systems of the improvement of cereal and grass crops. J. Plant. Physiol. 128:193-218.
White J, Chang S Y, Bibb M, Bibb M (1990) A cassette containing the bar gene of S. hygroscopicus: a selectable marker for plant transformation. Nucl. Acids Res 18:1062.
Wilson T (1999) Untranslated eader sequences from RNA viruses as enhancers of translation. U.S. Pat. No. 5,891,665.
Yammamoto T, G K Powell (1993) Bacillus thuringiensis Crystal proteins: Recent Advances in understanding its insecticidal activity. In: Advanded Engineered Pesticides. Ed. Leo Kim. P3-42. New York. 430p.

Zhao Z-Y, Gu W, Cai T, Tagliani L, Hondred D, Bond D, Schroeder S, Rudert M, Pierce D (2001) High throughput genetic transformation of maize mediated by Agrobacterium tumefaciens. Mol Breed 8:323-333.

1. A CODON-OPTIMIZED CRY1DA NUCLEIC ACID MOLECULE characterized in that it comprises a nucleic acid sequence having at least 70% similarity with the sequence of SEQ ID NO: 1.

2. The MOLECULE of claim 1, characterized in that it comprises a nucleic acid sequence having at least 90% similarity with the sequence of SEQ ID NO: 1.

3. The MOLECULE of claim 2, characterized in that the sequence is defined as SEQ ID NO: 1.

4. A NUCLEIC ACID CONSTRUCT, characterized in that it comprises the nucleic acid molecule, as defined in claim 1.

5. The CONSTRUCT of claim 4, characterized in that it further comprises a promoter sequence operably linked to said nucleic acid molecule.

6. The CONSTRUCT of claim 5, characterized in that the promoter sequence is maize ubiquitin gene (ubi) promoter sequence.

7. The CONSTRUCT of claim 4, characterized in that it further comprises a 3′ UTR terminator sequence.

8. The CONSTRUCT of claim 7, characterized in that the 3′ UTR terminator sequence is nopaline synthase (nos) gene terminator sequence.

9. The CONSTRUCT of claim 4, characterized in that it further comprises a selection gene, operably linked to at least one promoter sequence and at least one terminator sequence.

10. The CONSTRUCT of claim 9, characterized in that the promoter sequence is duplicated CaMV 35S gene promoter sequence from cauliflower mosaic virus and the terminator sequence is Tvsp gene terminator sequence that codes for soybean vegetative storage protein.

11. The CONSTRUCT of claim 4, characterized in that it further comprises other regulatory sequences.

12. The CONSTRUCT of claim 4, characterized in that it further comprises the nucleic acid sequence of SEQ ID NO: 10.

13. A VECTOR, characterized in that it comprises the nucleic acid molecule, as defined in claim 1.

14. A HOST CELL, characterized in that it comprises the nucleic acid molecule, as defined in claim 1.

15. A PLANT CELL, characterized in that it comprises the nucleic acid molecule, as defined in claim 1.

16. A TRANSGENIC PLANT, characterized in that it comprises the nucleic acid molecule, as defined in claim 1.

17. A CELL TRANSFORMATION METHOD, characterized in that it comprises introducing into said cell the nucleic acid molecule, as defined in claim 1.

18. The METHOD of claim 17, characterized in that the nucleic acid molecule integrates into the cell genome.

19. A METHOD OF PRODUCING A TRANSGENIC PLANT, characterized in that it comprises transforming the plant cell with the nucleic acid molecule, as defined in claim 1.

20. The METHOD of claim 19, characterized in that it further comprises selecting the plant cell transformed with the nucleic acid molecule, as defined in claim 1.

21. The METHOD claim 19, characterized in that it further comprises regenerating the transgenic plant from said plant cell.

22. The METHOD of claim 19, characterized in that the transgenic plant is resistant to crop pests.

23. The METHOD of claim 19, characterized in that the transgenic plant is a monocotyledonous plant.

24. The METHOD of claim 23, characterized by the monocotyledonous plant being a maize, rice, sugarcane, sorghum, wheat or brachiaria plant.

25. The METHOD of claim 22, characterized in that the crop pest is an insect.

26. The METHOD of claim 25, characterized in that the insect is of the order Lepidoptera.

27. The METHOD of claim 26, characterized in that the insect is Spodoptera frugiperda.

28. The METHOD of claim 26, characterized in that the insect is Diatrea saccharalis.

29. A METHOD OF CONTROLLING INVERTEBRATE PESTS IN CROP PLANTS, wherein the crop plants comprise the nucleic acid molecule, as defined in claim 1, characterized in that it comprises planting seeds obtained from a plant comprising the nucleic acid molecule, as defined in claim 1, in an area of cultivation of crop plants susceptible to invertebrate pests.

30. USE OF THE NUCLEIC ACID MOLECULE, as defined in claim 1, characterized in that it is for the production of a transgenic plant.

31. The USE of claim 30, characterized in that the transgenic plant is a monocotyledonous plant.

32. The USE of claim 31, characterized in that the monocotyledonous plant is a maize, rice, sugarcane, sorghum, wheat or brachiaria plant.

33. The USE of claim 30, characterized in that the transgenic plant is resistant to invertebrate pests.

34. USE OF THE NUCLEIC ACID MOLECULE, as defined in claim 1, characterized in that it is for controlling invertebrate pests.

35. The USE of claim 34, characterized in that the invertebrate pests are insects.
